(19) United States
(12) Patent Application Publication
(10) Pub. No.: US 2017/0175084 A1
(43) Pub. Date: Jun. 22, 2017

(54) TUMOR STEM CELLS

(71) Applicant: THE BOARD OF TRUSTEES OF THE UNIVERSITY OF ILLINOIS, Urbana, IL (US)

(72) Inventor: James A. RADOSEVICH, Belvidere, IL (US)

(73) Assignee: THE BOARD OF TRUSTEES OF THE UNIVERSITY OF ILLINOIS, Urbana, IL (US)

(21) Appl. No.: 15/452,138

(22) Filed: Mar. 7, 2017

Related U.S. Application Data
(63) Continuation of application No. 13/630,774, filed on Sep. 28, 2012, now abandoned.
Application No. 13/630,774 is a continuation of application No. 13/118,760, filed on May 31, 2011, now abandoned.
(60) Provisional application No. 61/396,527, filed on May 29, 2010.

(51) Int. Cl. C12N 5/095; C12N 5/00

(52) U.S. Cl. CPC C12N 5/0695; C12N 5/0093; C12N 2501/03; C12N 2506/23; C12N 2506/246; C12N 2506/30

(57) ABSTRACT

Tumor stem cells can be obtained by culturing a tumor cell population, and exposing the cultured tumor cell population to free radicals. In certain embodiments, the free radical agent can be a nitric oxide (NO) donor. In one embodiment, the free radical agent can be Diethylenetriamine NONOate (DETA NONOate) or agents that constitutively increase cellular nitric oxide, such as phosphodiesterase inhibitors or L-arginine, or agents that increase NO synthase in the population. The methods can further include inducing stem cells present in the population to expand and/or inducing dedifferentiation of tumor cells into tumor stem cells. Additionally, the present invention provides methods of selecting stem cells from a tumor cell population. Another aspect provides methods of screening for anti-tumor stem cell teherapeutic compounds by providing high nitric oxide (HNO) tumor cells, exposing the HNO cells to at least one compound, assessing one or more indicators of HNO cell health and determining toxicity of the compound to HNO tumor cells.

### REFERENCE TO RELATED APPLICATION

This application is a Continuation of application Ser. No. 13/630,774 filed on Sep. 28, 2012. Application Ser. No. 13/630,774 is a Continuation of application Ser. No. 13/118,760 filed on May 31, 2011. Application Ser. No. 13/118,760 claims the benefit of U.S. Provisional Application 61/396,527 filed on May 29, 2010. The entire contents of these applications are incorporated herein by reference in their entirety.

### TECHNICAL FIELD

The technical field of this invention is the study and treatment of cancer.

### BACKGROUND OF THE INVENTION

Cancer is one of the world's most significant epidemiological problems. While many advances in treatments have been made, cancers frequently recur and patients die of their disease. The tumor stem cell theory sets forth the premise that the recurrence of cancer is due to a very small population of “stem cells” (as low as 0.005%), which survive initial treatments. These stem cells are proposed to be both self-renewing and able to generate replicating, non-self-renewing “tumor cells.” The tumor cells are thought to make up the bulk of the tumor, and are proposed to be the cells that are most sensitive to various treatments, giving rise to the initial tumor response. Treatments therefore act as selective pressure to enrich tumor stem cell populations. The stem cell model advocates that the tumor stem cells need to be targeted to cure patients.

Experimental data supports the tumor stem cell theory by showing that as few as 5 tumor stem cells (isolated by flow cytometry activated cells sorting, FACS) could give rise to a tumor in mice. This, as compared to several million tumor cells from the same cell line that were also FACS sorted (no tumor stem cells), did not give rise to palpable tumors.

Clinical and experimental data suggest that patients presenting with (or converting to), tumors with high levels of nitric oxide (NO) tended to do more poorly than those patients which had low expressing tumors. Nitric oxide has been found to play a role in promoting solid tumor growth and progression. For example, nitric oxide generation by inducible nitric oxide synthase (iNOS) has been implicated in the development of multiple cancers. In fact, it has been suggested that tumor cells producing or exposed to low levels of nitric oxide, or tumor cells capable of resisting nitric oxide-mediated injury undergo a clonal selection because of their survival advantage.

A major problem in the field of tumor stem cell biology is the lack of cells to be studied. This is further complicated by the fact that, to date, these tumor stem cells must be FACS sorted. To make advances more difficult, even if one does sort a pure population of tumor stem cells, as soon as they are placed in culture, they begin generating tumor cells, and the population of tumor stem cells quickly results in a greatly diluted tumor stem cell population, ending up with what one started with before FACS sorting.

Accordingly, there exists a need for better techniques for isolating tumor stem cells and for utilizing such isolated tumor stem cells for the study and treatment of cancer.

### SUMMARY OF THE INVENTION

Tumor stem cell enrichment methods are disclosed whereby tumor stem cells can be obtained by culturing a tumor cell population, and exposing the cultured tumor cell population to free radicals. In certain embodiments, the free radical agent can be a nitric oxide (NO) donor. In one preferred embodiment, the NO donor can be Diethylenetriamine NONOate (DETA NONOate). Alternatively, the free radicals can be provided by agents that constitutively increase cellular nitric oxide, such as phosphodiesterase inhibitors or L-arginine, or by agents that increase NO synthase in the population. The methods can further include inducing stem cells present in the population to expand and/or inducing dedifferentiation of tumor cells into tumor stem cells.

Additionally, the present invention provides methods of selecting stem cells from a tumor cell population. For example, the stem cells can be isolated by exposing the population to a level of free radicals sufficient to selectively kill tumor cells but not stem cells. Alternatively, the stem cells can be isolated by cell sorting techniques, for example by exclusion of a vital dye, such as Hoechst 33342, or by expression of a marker, such as aldehyde dehydrogenase (ALDH). Identification and/or isolation of stems cells can also be carried out by assay techniques, e.g., based on exhibition of shorter tails in a COMET assay or upregulation of a DNA repair enzyme, such as the APE-1 DNA repair enzyme.

In one approach, very low amounts of a nitric oxide donor were added to the culture medium of a tumor cell population, every 2-3 days, and incrementally increased over a period of 6-24 months. This approach has successfully produced ten cell line sets (parent and high nitric oxide adapted) where the High Nitric Oxide (HNO) cell lines thrive in a concentration of nitric oxide donor that is lethal to the parent. These cell lines have been maintained for over two years. Table 4 presents a summary table of the cell cultures, and indicates that the HNO cell lines are much more aggressive in their biological properties. This is consistent with what one would expect for an enriched population of tumor stem cells. In addition, the HNO cell lines (and not the parent cell lines) that have been tested to date, express two properties that have been used to define tumor stem cells: 1) they are positive for expression of Aldehyde Dehydogenase-1 (ALDH-1), and 2) they have enriched populations of Hoechst 33342 negative populations (as high as 50%).

Thus the “nitric oxide selective pressure model” of the present invention has enriched a population of cells that have tumor stem cells “like” properties in the HNO cultures.

Contrary to the current stem cell theory, tumor cells can be converted to tumor stem cells, upon long-term exposure to nitric oxide, other nitrogen free radicals, hydrogen peroxide, other oxygen based free radicals and other types of free radicals. The exposure can be at increased doses, varying doses, or constant doses.

As shown by immunocytochemistry that these tumor stem cells “like” cell lines abundantly express Aldehyde Dehydrogenase-1 (ALDH-1), that ALDH-1 activity is up-regulated in these cells, and some of the cell lines tested to date, had up to 50% of the cells expressing no staining properties for the vital dye, Hoechst 33342. Both of these markers have been reported as being properties that are uniquely expressed be tumor stem cells. By using a number of other biomarkers and cellular assays, a compelling case is presented that these are in fact tumor stem cells (See Table 1 Below).

Moreover, if the selective pressure of exogenous nitric oxide is removed, that the cell populations (over time) have been found to revert to that same ratios of tumor stem cells and tumor cells originally present in the population prior to enrichment. This further demonstrates that nitric oxide exposure does not simply up regulate ALDH-1, and makes the population more resistant to vital dye uptake, without any direct change from tumor cells to tumor stem cells. Gene chip experiments likewise show a profile of the genes that are up regulated and down regulated for one tumor cell line pair (A549 vs. HNO A549) and therefore show a markedly different gene profile for the tumor stem cells derived from A549. Other tumor stem cell lines have similar modified profiles.

The methods and compositions disclosed herein generally relate to tumor stem cells. In one aspect, a method of obtaining the tumor stem cells is disclosed. The method includes the steps of culturing a cell population and exposing the cultured cell population to free radicals. The free radicals can include superoxides, nitric oxide, nitrogen-based free radicals, hydrogen peroxide, other oxygen-based free radicals and other types of free radicals. In an exemplary embodiment, the free radical is a nitric oxide. In another embodiment the free radical is a nitric oxide donor. Examples of nitric oxide donors can include nitric oxide donors can include DETANONOate (DETANONO, NONOate or 1-substituted diazen-1-ium-1,2-diolate compounds containing the [N(O)NO]—functional group: DEA/NO; SPER/NO; DETA/NO; OXI/NO; SULFI/NO; PAPA/NO; MAHMA/NO and DPTA/NO), PAPANONOate, SNAP (S-nitroso-N-acetylpenicillamine), sodium nitroprusside, sodium nitroglycerine, sildenafil, atorvastatin, compounds which increase nitric oxide, phosphodiesterase inhibitors, L-arginine, effectors of nitric oxide synthase, and nitric oxide mimetics. In another embodiment, the free radical increases nitric oxide synthase, such as inducible nitric oxide synthase (iNOS).

In another aspect, exposing the cultured cell population to free radicals includes increasing the concentration of free radicals. The exposure of the cultured cell population to the free radical can be at increased doses, varying doses, or constant doses.

The method can also include the steps of culturing a tumor cell population and exposing the cultured tumor cell population to free radicals. In another embodiment, the cell population can be normal cells, non-tumor cells, cell lines, primary tissues, and immortalized cells and the cultured normal cell, non-tumor cell, cell line, primary tissue, and immortalized cell populations can be exposed to free radicals.

The method also includes increasing a population of tumor stem cells. The tumor stem cells can be induced to expand by the exposure to free radicals. Moreover, exposing the cultured tumor cell population to free radicals can selectively expand the tumor stem cells over the tumor cells, thereby increasing the concentration of tumor stem cells in the cultured tumor cell population. Exposing the cultured tumor cell population to concentrations of free radicals can also selectively kill the tumor cells, thereby increasing the number of tumor stem cells in the cultured tumor cell population. In addition, exposing the cultured tumor cell population to concentrations of free radicals can dedifferentiate the tumor cells to tumor stem cells, thereby increasing the number of tumor stem cells in the cultured tumor cell population.

The tumor stem cells can be isolated or identified from the tumor cells. Staining the cultured tumor cell population with a vital dye, such as Hoechst 33342, can be used to exclude non-tumor stem cells, stain positively for Hoechst 33342, from tumor stem cells, negative staining with Hoechst 33342.

Tumor stem cells can also be isolated or identified through expression of proteins. Cell surface marker expression of the cultured tumor cell population can be used to isolate or identify the tumor stem cell population from the tumor cells. Surface makers can include, but are not limited to, CD24, CD34, CD38, CD44, CD117, CD133, CD166. Protein expression can also be measure. Proteins such as aldehyde dehydrogenase (ALDH-1), glutathione S-transferase-pi (GST-π), inductible nitric oxide synthase (iNOS) and apurinic/apyrimidinic endonuclease-1 (APE-1) can be used to isolate or identify the tumor stem cell population from the tumor cells. For example, increased in expression of ALDH-1 or upregulation of APE-1 in tumor stem cells can be used to differentiate the cells from tumor cells.

Tumor stem cells can also be isolated or identified through DNA content assays. Assays that determine the amount of DNA fragmentation found in a single cell, such as COMET assays, can be used to identify tumor stem cells.

In one aspect, a high nitric oxide (HNO) tumor stem cell is disclosed. The HNO tumor stem cell can exhibit one or more characteristics including, but not limited to, cell surface expression of at least the cell surface markers CD38 and CD166, increased expression of aldehyde dehydrogenase (ALDH) and upregulation of at least one DNA repair enzyme. Moreover, the HNO tumor stem cell can also be characterized by one or more of the characteristics of resistance to DNA fragmentation, growth in high free radical environments, resistance to UV and gamma radiation and temperature insensitivity.

In one embodiment, a gene expression profiled for the HNO tumor stem cell can be determined. The gene expression profile of the HNO tumor stem cell can be the same or similar to the expression profile shown in Appendix A. In another embodiment, the expression profile of the HNO tumor stem cells can be about 50% homologous, about 60% homologous, about 70% homologous, about 75% homologous, about 80% homologous, about 85% homologous, about 90% homologous, 91% homologous, 92% homologous, 93% homologous, 94% homologous, about 95% homologous, 96% homologous, 97% homologous, 98% homologous, about 99% homologous or 100% homologous to the expression profile shown in Appendix A. Moreover, the gene expression profile of the HNO tumor stem cells can be at least or about 50% homologous to a subset of genes in the expression profile as shown in Appendix A. In yet another embodiment, a subset of genes can include as set of 5 or more genes.

In another aspect, methods of screening to identify compound that can inhibit, if not eliminate, tumor stem cell populations is disclosed. The screening methods can include providing high nitric oxide (HNO) tumor cells, exposing the HNO cells to at least one compound, assessing one or more indicators of HNO cell health and determining toxicity of the compound to HNO tumor cells.

The compounds screened can accelerate the degradation of NO, inhibit the effects of NO, inhibit the production of NO synthase, generate or release endogenous or exogenous NO inhibitors, or inhibit or prevent NO utilization by the cell. The compounds can include nitric oxide inhibitors, nitric oxide antagonists, small molecule superoxide dismutase mimetics, superoxide dismutase agonists, inhibitors of hydroxyl radicals, inhibitors of superoxide anions and free radical scavengers. In an exemplary embodiment, the compound is a nitric oxide inhibitor. Examples of nitric oxide inhibitors can include NG,NG-dimethylarginine (asymmetrical dimethylarginine, ADMA), NG-nitro-L-arginine methyl ester (L-NAME), aminoguanidine, nitro-L-arginine, N omega-nitro-L-arginine, N omega-monomethyl-L-arginine, 2-phenyl-4,4,5,5-tetramethylimidazoline-1-oxyl 3-oxide (PTIO), carboxy-PTIO, and carboxymethoxy-PTIO.

In another embodiment, the compound is a free radical scavenger. The free radical scavenger can include curcumin, diacetylcurcumin, inhibitors of superoxide anions, epicatechin gallate, epigallocatechin gallate, gallocatechin, gallocatechin gallate, lipoic acid, tocopherol, hydroxytyrosol, ascorbic acid, balsalazide, caffeic acid, caffeic acid phenethyl ester, chlorogenic acid, chlorphyllin, delphinidin chloride, diosmin, ellagic acid, eugenaol, ferulic acid, fucoxanthin, gallic acid, ginkgolide B, herperidin, kaempferol, linoleic acid, luteolin, lycopene, N-acetyl-L-cysteine, oleic acid, resveratrol, rutin hydrate, se-(methyl)selenocysteine hydrochloride, seleno-L-methionine, sodium selenite, xanthophyll, carotene, courmaric acid, and salts and derivatives thereof.

In yet another embodiment, the compound is an antibody. The antibody can be monoclonal antibody, a polyclonal antibody, bi-specific antibody, a humanized antibody, a chimeric antibody, an anti-idiopathic (anti-ID) antibody, a single-chain antibody, a Fab fragment, a F(ab′) fragment, and a fusion protein. Moreover, the antibody can be directed against an antigen specific for at least one selected from the group consisting of tumor cells, tumor stem cells, NO, NOS, inductible nitric oxide synthase (iNOS), aldehyde dehydrogenase (ALDH-1), glutathione S-transferase-pi (GST-π), and apurinic/apyrimidinic endonuclease-1 (APE-1). In an exemplary embodiment, the antibody is directed against the tumor stem cells.

Exposing the HNO tumor cells to varying concentrations of candidate therapeutic agents can also impact the toxicity of the compound to HNO tumor cells. In an exemplary embodiment, the concentration of the compound is varied until one or more indicators of HNO cell health is altered. Examples of final growth media compound concentrations can range in concentration from about 0.05 micromolar, 0.1 micromolar, 1.0 micromolar, 5.0 micromolar, 10.0 micromolar, 20.0 micromolar, 50.0 micromolar, 100 micromolar, to about 300 micromolar of the compound in culture media. Moreover, altered indicators of HNO cell health can include alteration in cell viability, altered cell surface marker expression, reduced tumorigenicity, stem cell ratio to tumor cells, chemotherapy sensitivity, temperature sensitivity, protein expression, and DNA degradation.

The HNO cells used to screen compounds can be generated by the methods disclosed to obtain the tumor stem cells. To determine efficacy or toxicity of the compounds to the HNO tumor cells one or more health indicators can be assessed. Examples of HNO cell health indicators can include cell viability, cell surface marker expression, tumorigenicity, stem cell ratio to tumor cells, chemotherapy sensitivity, temperature sensitivity, protein expression, and DNA degradation. Moreover, expression of certain proteins, such as aldehyde dehydrogenase (ALDH-1), glutathione S-transferase-pi (GST-π), inductible nitric oxide synthase (iNOS) or apurinic/apyrimidinic endonuclease-1 (APE-1) can also be assessed.

In one embodiment, determining the HNO cell health indicators can include measuring tumorigenicity of the HNO cells. Tumorigenicity can be measured through one or more assays such as contact inhibition, serum free growth, migration assays and angiogenesis. Examples of reduced tumorigenicity can be characterized by contact inhibition, lack of or reduced serum free growth, inability to or reduced migration in migration assays, reduced or lack of angiogenesis.

The candidates can also be screened in both the HNO cells and their parent cells for comparison. This would include providing parent cells to the high nitric oxide (HNO) tumor cells, exposing the parent cells to the at least one compound, assessing one or more indicators of parent cell health, determining toxicity of the compound to parent cells and comparing the toxicity of the compound the HNO tumor cells. Moreover, screened compounds can be selected for higher toxicity to HNO tumor cells than parent cells, then administered with a pharmaceutically acceptable carrier to a subject and the efficacy of the screened compounds can be determined in the subject.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIG. 1 shows the adaptation curve for head and neck cancer cell lines SCC016 and SCC040;

FIG. 2 shows the adaptation curve for head and neck cancer cell lines SCC056 and SCC116;

FIG. 3 shows the adaptation curve for lung adenocarcinoma cell line A549 and mouse tumor cell line LP07;

FIG. 4 shows adaptation curves of the four breast cancer cell lines (BT-20, MCF-7, T-47D, and Hs578T) that were treated with increasing concentrations of the NO donor DETA-NONOate over time;

FIG. 5 shows graphs of MTT viability/proliferation of the five HNSCC parent and HNO tumor cells in standard growth media supplemented with 600 μM DETA-NONOate. Data shown as the mean normalized absorbance±standard error (n=3; *P<0.05 versus parent cells, **P<0.01 versus parent cells);

FIG. 6 shows MTT proliferation/viability curves for A549 and LP07 parent and HNO cell lines in standard growth media supplemented with 300 μM (LP07-HNO) or 600 μM (A549-HNO) DETA-NONOate;

FIG. 7 shows graphs of MTT viability/proliferation curves of four sets of parent and HNO breast tumor cells in standard growth media supplemented with 600 μMDETA-NONOate;

FIG. 8 is a graph showing a MTT proliferation/viability assay of A549 parent and A549-HNO cells in serum-less media supplemented with 600 μM DETA-NONOate;

FIG. 9 shows graphs of MTT viability/proliferation of the SCC016 and SCC040 HNSCC parent and HNO tumor cells on soft agar supplemented with 600 μM DETA-NONOate;

FIG. 10 shows graphs of MTT viability/proliferation of the SCC056, SCC114 and SCC116 HNSCC parent and HNO tumor cells on soft agar supplemented with 600 μM DETA-NONOate;

FIG. 11 shows graphs of MTT viability/proliferation of the A549 and LP07 parent and HNO tumor cells on soft agar supplemented with 600 μM DETA-NONOate;

FIG. 12 shows graphs of MTT viability/proliferation curves of SCC016 and SCC040 parent and HNO head and neck tumor cells exposed to varying concentrations of hydrogen peroxide for 24 h with media supplemented with 600 μM DETA-NONOate. Data are shown as the mean normalized absorbance±standard error (n=3; *P<0.05 versus parent cells, **P<0.01 versus parent cells);

FIG. 13 is a graph of MTT viability/proliferation of SCC058 parent and HNO head and neck tumor cells exposed to varying concentrations of hydrogen peroxide for 24 h with media supplemented with 600 μM DETA-NONOate. Data are shown as the mean normalized absorbance±standard error (n=3; *P<0.05 versus parent cells, **P<0.01 versus parent cells);

FIG. 14 shows graphs of MTT viability/proliferation curves of SCC114 and SCC116 parent and HNO head and neck tumor cells exposed to varying concentrations of hydrogen peroxide for 24 h with media supplemented with 600 μM DETA-NONOate. Data are shown as the mean normalized absorbance±standard error (n=3; *P<0.05 versus parent cells, **P<0.01 versus parent cells);

FIG. 15 shows graphs of MTT proliferation/viability assays of A549 and LP07 parent and HNO cells in response to a hydrogen peroxide dilution series in media supplemented with 300 μM (LP07-HNO) or 600 μM (A549-HNO) DETA-NONOate;

FIG. 16 shows graphs of MTT viability/proliferation curves of Hs578T and MCF-7 parent and HNO breast tumor cells exposed to varying concentrations of hydrogen peroxide for 24 h in media supplemented with 600 μM DETA-NONOate;

FIG. 17 shows graphs of MTT viability/proliferation curves of BT-20 and T-47D parent and HNO breast tumor cells exposed to varying concentrations of hydrogen peroxide for 24 h in media supplemented with 600 μM DETA-NONOate;

FIG. 18 shows graphs of cell viability of SCC016, SCC040 and SCC056 parent and HNO HNSCC cells, as measured by the DPA assay, 96 h after exposure to varying doses (0-28 Gy) of X-ray radiation while being cultured in media supplemented with 600 μM DETA-NONOate. Data are presented as mean normalized absorbance ±standard error (n=3). *P<0.05 versus parent cells, **P<0.01 versus parent cells;

FIG. 19 shows graphs of cell viability of SCC016, SCC040 and SCC056 parent and HNO HNSCC cells, as measured by the MTT assay, 72 h after exposure UV radiation for varying amounts of time (0-10 min) while being cultured in media supplemented with 600 μM DETA-NONOate. Data are presented as mean normalized absorbance±standard error (n=4 for SCC016 parent/HNO; n=3 for SCC040 parent/HNO and SCC056 parent/HNO). **P<0.01 versus parent cells;

FIG. 20 is a bar graph showing Comet tail length or DNA strand breaks, as measured by single cell gel electrophoresis, in parent and HNO tongue squamous carcinoma cell lines. Data are presented as the mean Comet tail length±standard error (n=15 for SCC016 parent/HNO and SCC040 parent/HNO; n=20 for SCC056 parent/HNO). *P<0.05 versus parent cells; **P<0.01 versus parent cells; and

FIG. 21 show a bar graph of relative expression of iNOS expression in parent (P) and HNO tongue squamous carcinoma cell lines. Western blot analysis shown below the bar graph standardized against b-actin expression. Data are presented as the average relative expression level±standard error (n=3).

### DETAILED DESCRIPTION

The compositions and methods described herein utilize tumor stem cells that have adapted by certain nitric oxide-mediated. The compositions and methods are also directed to nitric oxide-blocking drugs that may be useful in treating certain human cancers.

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

All publications, patents and patent applications cited herein, whether supra or infra, are hereby incorporated by reference in their entirety. As used in this specification and the appended claims, the singular forms “a,” “an,” and “the” include plural references unless the content clearly dictates otherwise. The terms used in this invention adhere to standard definitions generally accepted by those having ordinary skill in the art. In case any further explanation might be needed, some terms have been further elucidated below.

Nitric oxide is an efficient hypoxic radiosensitizer, as it may mimic the effects of oxygen on fixation of radiation-induced DNA damage. Within tumors, even the endogenous production of NO by the enzyme nitric oxide synthase (NOS) is capable of radiosensitizing tumor cells. Previously it was regarded that high levels of nitric oxide, when induced in certain cells, can cause cytostasis and apoptosis. For example, it has been shown that exposure to high levels of nitric oxide to be an exploitable phenomenon to promote death in murine K-1735 melanoma cells (J. Exp. Med. 1995 181:1333-1343). In addition, WO 93/20806 discloses a method of inducing cell cytostasis or cytotoxicity by exposing cells to a compound such as spermine-bis(nitric oxide) adduct monohydrate at 500 μM which is capable of releasing nitric oxide in an aqueous solution.

U.S. Pat. No. 5,840,759, U.S. Pat. No. 5,837,736, and U.S. Pat. No. 5,814,667, also disclose methods for using mg/kg quantities of nitric oxide releasing compounds to sensitize hypoxic cells in a tumor to radiation. These patents disclose methods of using the same nitric oxide-releasing compounds at mg/kg levels to protect non-cancerous cells or tissue from radiation, to sensitize cancerous cells to chemotherapeutic agents, and to protect non-cancerous cells or tissue from chemotherapeutic agents.

However, it has been discovered that patients with tumors with high levels of NO tended to do more poorly than those patients which had low NO expressing tumors. High levels of NO and increased expression of nitric oxide synthase (NOS) have been implicated in tumor progression. These findings suggest that NO may play multiple roles depending on whether it is present in the microenvironment at high or low concentrations. Three main isoforms of NOS have been identified: inducible (iNOS), endothelial constitutive (eNOS), and neural (nNOS).

Multiple human cell types have been shown to produce NO. Moreover, human tumor cells overexpress endothelial constitutive nitric oxide synthase (ecNOS) in human head and neck primary cancers. The term “head and neck” as used herein applies to tumors that arise in the nasal and paranasal cavities, nasopharynx, orophaynx, oral cavity, larynx and hypopharynx. While keenly aware of the differences among the individual tumor sites, NOS/NO expression is similar among the varied sites.

As shown herein, over or aberrant production of NO leads to the undesirable toxic effects noted in a broad range of tissues, whereas low levels of NO seem to provide good and useful signaling in an equally large number of tissues. It is for this reason that NO is thought to be a double-edged sword with both beneficial and adverse properties, depending on the situation and the environment. Also as demonstrated herein, NO and other reactive nitrogen species play a crucial role in tumorigenesis and/or tumor suppression. Recent reports demonstrate that NO-derived nitrogen oxides can interact with DNA molecules leading to carcinogenic aducts. Peroxydation, alkylation, deamination, or direct oxidation of DNA are also possible mechanisms. Utilizing reagents and cell lines to produce a clear understanding of the roles NO plays in human tumors are key in the design of new therapeutic approaches for the treatment of these tumors.

Overexpression of at least one NOS isoform, as well as nitrotyrosine (a marker of NOS activity), are seen in a variety of carcinomas, including head and neck, salivary, and esophageal tumors. A correlation between iNOS expression in human oral squamous cell carcinomas and cervical lymph node involvement is also noted. Additionally, enhanced expression of iNOS is observed with the development of experimentally induced hamster oral carcinoma.

Given the wide spectrum of NO expression found in patients, it is critically important to understand how cancer cells react to various levels of NO exposure over long periods of time. However, studying this phenomenon in a clinical setting has been extremely difficult given the lack of cells that mimic the tumors, virtually eliminating the possibility of designing new therapeutic approaches for the treatment of these tumors.

Production of High Nitric Oxide (HNO) Cells

High Nitric Oxide adapted tumor cell lines are greatly enriched for tumor stem cells. Contrary to the current stem cell theory, tumor cells can be converted to tumor stem cells upon long term exposure to Nitric Oxide (NO), other nitrogen free radicals, hydrogen peroxide, other oxygen based free radicals and other types of free radicals.

As used herein, the terms “HNO stem cell,” “HNO tumor stem cell,” “tumor stem cell,” “HNO adapted tumor cell,” “HNO adapted tumor stem cell” are used interchangeably to refer to a self-renewing tumor cell that can reproduce indefinitely to form the bulk of a tumor, or tumor cells. A stem cell can divide to produce two daughter stem cells, or one daughter stem cell and one progenitor (“transit”) cell, which then proliferates into the tumor's non-proliferative tumor cells. The “tumor stem cell” used herein includes “tumor progenitor cells” unless otherwise noted. The term “pluripotential”, “pluripotential for differentiation” or “pluripotent” can also refer to a cell that is positive for one or more of the pluripotent markers and the tumor stem cell has the potential to produce additional tumor stem cells or differentiate to tumor cells or non-self renewing tumor cells.

Different cell carcinomas have been exemplified in the Examples as producing HNO adapted tumor stem cells. Such examples can include, but are not limited to, head and neck region tumors, squamous cell tumors, human and mouse lung cancer cell lines and breast cancer cell lines. The methods and compositions described herein can also include a broader range of tumorigenic cell lines (human- and other mammal-derived), primary tumor tissues, non-tumorigenic cell lines (human- and other mammal-derived) and other cells types. The breast cancer cell lines used herein can be also broadly divided into two groups, estrogen positive, and estrogen negative. Both can be use use in the methods and compositions. Similarly, lung cancer cell lines, for the most part, can be divided into small and non-small cancers, with adenocarcinomas being the most frequent non-small subtype. Hematopoietic cells can also be used in the methods and compositions disclosed.

Any type of human or animal cells can be useful in the methods and compositions disclosed herein. In one embodiment, normal cells, non-tumor cells, tumor cells, cell lines, primary tissues, and immortalized cells (eg: SV-40 transformed) can be adapted to have stem cell properties by exposing them to long term (constant of increasing) free radicals. In another embodiment, the normal cells, non-tumor cells, tumor cells, cell lines, primary tissues, and immortalized cells are adapted to produce the tumor stem cell population.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As disclosed herein, the term “free radical” refers to atoms, molecules or ions with unpaired electrons. The unpaired electrons are highly chemically reactive. Some examples of free radicals can include superoxides, nitric oxide, nitrogen free radicals, hydrogen peroxide, other oxygen based free radicals and other types of free radicals.

Moreover, nitric oxide donors are also included in the methods and compositions to increase free radical concentrations within the cells. The term “nitric oxide donor” as used herein is intended to encompass any compound which is able to donate nitric oxide or promote an increase in nitric oxide. There are families of compounds which donate nitric oxide. Included among these compounds are: DETANONOate (DETANONO, NONOate or 1-substituted diazen-1-ium-1,2-diolate compounds containing the [N(O)NO]—functional group: DEA/NO; SPER/NO; DETA/NO; OXI/NO; SULFI/NO; PAPA/NO; MAHMA/NO and DPTA/NO), PAPANONOate, SNAP (S-nitroso-N-acetylpenicillamine), sodium nitroprus side, sodium nitroglycerine, sildenafil, and atorvastatin. Compounds which promote the increase in nitric oxide include phosphodiesterase inhibitors, L-arginine, effectors of nitric oxide synthase, and nitric oxide mimetics.

The cells can be cultured prior to exposure to free radicals. The cells can be cultured in suspension or as adhered to a substrate. By “suspension” or “suspension culture” is meant a cell culture maintained in a liquid. Although not required, suspension cultures are frequently maintained in suspension by stirring or shaking or other means of agitation. The term “adherent culture” refers to cells that are maintained adhered to a substrate.

Cell culture media provide the nutrients necessary to maintain and grow cells in a controlled, artificial and in vitro environment. Characteristics and compositions of the cell culture media vary depending on the particular cellular requirements. Important parameters include osmolarity, pH, and nutrient formulations. The requirements of cell culture in vitro can comprise, in addition to basic nutritional substances, a complex series of growth factors. Usually, these are added to the culture medium by supplying it with animal sera or protein-fractions from animal sources. Typically, cell culture media formulations are supplemented with a range of additives, including undefined components such as fetal bovine serum (FBS) (10-20% v/v) or extracts from animal embryos, organs or glands (0.5-10% v/v). While FBS is the most commonly applied supplement in animal cell culture media, other serum sources are also routinely used, including newborn calf, horse and human. Other supplements can provide carriers or chelators for labile or water-insoluble nutrients; bind and neutralize toxic moieties; provide hormones and growth factors, protease inhibitors and essential, often unidentified or undefined low molecular weight nutrients; and protect cells from physical stress and damage.

The cultures can also be substantially “serum-free” or cultured in “serum-free media” or “SFM.” A number of SFM formulations are commercially available, such as those designed to support the culture of endothelial cells, keratinocytes, monocytes/macrophages, lymphocytes, hematopoietic stem cells, fibroblasts, chondrocytes or hepatocytes which are available from Life Technologies, Inc. (Rockville, Md.). Often used interchangeably with “defined culture media”. SFM are media devoid of serum and some protein fractions (e.g., serum albumin). Indeed, several SFM that have been reported or that are available commercially, including several formulations supporting in vitro culture of multiple cell types.

The cell cultures can be grown to optimal confluency prior to exposure to free radicals. In one embodiment, the cells can be maintained in culture without exposure to free radicals until the cells reach about 10%, 20%, 30%, 40%, 50%, 60%, 70%, 80%, 90% or about 100% confluency. In another embodiment, the cells are maintained in culture over a prolonged period of time. These cells are maintained as parent cells. In yet another embodiment, the parent cells are continuously passaged in media lacking or essential free of free radicals.

The exposure of the cells to the free radical can be at increased doses, varying doses, or constant doses. For example, cells can be subjected to increased doses over a predetermined time. In one embodiment, the cells can be subjected to about a 5 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM or about 100 μM increase in dose of free radical. In another embodiment, the cells can be subjected to an increase in free radical about every hour, 2 hrs, 4 hrs, 6 hrs, 12 hrs, 18 hrs, 24 hrs, 36 hrs, 2 days, 3 days, 4 days, 5 days, 6 days, 7 days, 10 days, 12 days, 14 days, 16 days and about 20 days. In yet another embodiment, the increase in free radical exposure can be dependent on the time between each increase in free radical exposure. For example, the free radical can be increased about 50 μM approximately every 2-3 days or increased about 25 μM each day. In one embodiment, the cells are grown in the presence of free radical and the increase in free radical occurs when the cells reach about 10%, 20%, 30%, 40%, 50%, 60%, 70%, 80%, 90% or about 100% confluency in the presence of free radical. In another embodiment, the concentration of free radical in the culture media can be about 5μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 120 μM, 130 μM, 140 μM, 150 μM, 160 μM, 170 μM, 180 μM, 190 μM, 200 μM, 210 μM, 220 μM, 230 μM, 240 μM, 250 μM, 260 μM, 270 μM, 280 μM, 290 μM, 300 μM, 310 μM, 320 μM, 330 μM, 340 μM, 350 μM, 360 μM, 370 μM, 380 μM, 390 μM, 400 μM, 410 μM, 420 μM, 430 μM, 440 μM, 450 μM, 460 μM, 470 μM, 480 μM, 490 μM, 500 μM, 510 μM, 520 μM, 530 μM, 540 μM, 550 μM, 560 μM, 570 μM, 580 μM, 590 μM, 600 μM, 610 μM, 620 μM, 630 μM, 640 μM, 650 μM, 660 μM, 670 μM, 680 μM, 690 μM, 700 μM, 710 μM, 720 μM, 730 μM, 740 μM, 750 μM, 760 μM, 770 μM, 780 μM, 790 μM, 800 μM, 810 μM, 820 μM, 830 μM, 840 μM, 850 μM, 860 μM, 870 μM, 880 μM, 890 μM and 900 μM. In another embodiment, the cells are maintained at a constant dose of free radical over a prolonged period of time. In yet another embodiment, the cells are continuously passaged in media supplemented with constant dose of free radical.

Viability of the cells cultured in the presence of free radical can be assessed by measured routinely used. Such measures can include, chemical analysis, flow cytometry, trypan blue exclusion, ELISA, measuring metabolic activity, localization of proteins, localization of nucleic acids, measuring protein content and measuring nucleic acid content, membrane characteristics and other known assays used frequently in the art. Common assays used can include 3-(4,5-dimethylthiazol-2-yl)-2,5-diphenyltetrazolium bromide assays, Hoechst staining (33342 and 33258) of nucleic acids, propidium idodide staining of nucleic acids, measuring ATP contained in the cells, resazurin and formazan assays, methyl violet, TUNEL assays, flow cytometry of molecules found in viable or non-viable cells, such as mitochondrial surface markers, nucleic acids, and other known molecules and assays used to differentiate viable from non-viable cells.

The tumor stem cells can be characterized by cell surface markers, expression of genes, expression of proteins, regulation of cellular processes, etc. For example, cell surface marker expression can include any one or more of the following, CD24, CD34, CD38, CD44, CD117, CD133, CD166 and others. The tumor stem cells may express cell surface markers that are specific to the type of parent cell. For example, hematopoietic cells express CD45, lung cells express ALDH-1, epithelial cells express CD50 and CD44, etc. In addition, tumor stem cells may express cell surface markers that are specific for stem cells, such as CD34, CD117, etc.

The tumor stem cells can also be characterized by tumorigenicity, sensitivity to UV radiation and/or temperature changes. Tumorigenicity of the tumor stem cells can be assessed by methods commonly used. Common methods can include in vitro assays, such as contact inhibition, serum free growth, migration assays, angiogenesis assays, etc. Common methods can also include in vivo assays, such as tumor formation in animal models. In vivo growth of HNO cells treated with anti-NO therapy can include intravenous, intraperitoneal or subcutaneous injection of cells. The injections can include limiting dilutions of the tumor cells to determine efficacy of the anti-NO therapy in comparison to non-treated tumor cells.

In another embodiment, the tumor stem cells can be characterized by gene expression or protein expression. Specific genes or proteins may be analyzed, such as aldehyde dehydrogenase (ALDH-1), glutathione S-transferase-pi (GST-π), inductible nitric oxide synthase (iNOS) and apurinic/apyrimidinic endonuclease-1 (APE-1). In addition, global gene expression profiles can be compared with the tumor stem cells. Such expression profiles are exemplified in Appendix A.

Moreover, tumor stem cells can be homologous to the expression profile of Appendix A. The term “homology” or “identity” as used herein refers to the percentage of likeness between expression profiles. To determine the homology or percent identity of expression profiles, the expression patterns are aligned for optimal comparison purposes. The percent homology between the two expression profiles is a function of the number of identical genes shared by the cells under comparison, taking into account the number of genes, and the expression of each gene, which need to be introduced for optimal alignment of the two sequences.

Such homology can be, for example, at least 50% homology to Appendix A or a subset of genes shown in Appendix A. A subset of genes can include as set of one or more genes, 5 or more, 10 or more, 15 or more, 20 or more, 30 or more, 40 or more, 50 or more, 100 or more, 150 or more, 200 or more, 300 or more, 400 or more, 500 or more, 600 or more, 700 or more, 800 or more, 900 or more, 1000 or more, 1500 or more, 2000 or more, 2500 or more, 3000 or more, 3500 or more, 4000 or more, 4500 or more, 5000 or more, 5500 or more, 6000 or more, 6500 or more, 7000 or more, 7500 or more, 8000 or more, 8500 or more, 9000 or more, 9500 or more, 10,000 or more, 10,500 or more, 11,000 or more, 11,500 or more, 12,000or more, 12,500 or more, 13,000 or more, 13,500 or more, 14,000 or more, or 14,500 or more genes. Moreover, the expression profile of a gene or subset of genes can be at least about or about 50% homologous, about 60% homologous, about 70% homologous, about 75% homologous, about 80% homologous, about 85% homologous, about 90% homologous, 91% homologous, 92% homologous, 93% homologous, 94% homologous, about 95% homologous, 96% homologous, 97% homologous, 98% homologous, about 99% homologous or 100% homologous to the expression profile of the tumor stem cells thereof. Also disclosed is a stem cell that has at least 60% homology in expression profile of a gene or subset of genes as Appendix A. In another embodiment, the stem cell has at least about 50%, 60%, 70%, 80%, 85%, 90%, 91%, 92%, 93%, 94%, 95%, 96%, 97%, 98%, 99% or 100% homology in expression profile(s) of a gene or subset of genes as Appendix A.

Inhibition of NO

The present invention also relates to the suppressing nitric oxide adaptation and/or nitric oxide synthase to inhibit and prevent a tumor stem cell development and selection. The methods and formulations of the present invention provide new therapeutic approaches for the treatment and prevention of cancer in mammals.

For purposes of the present invention, by “treatment” or “treating” it is meant to encompass all means for controlling cancer by reducing growth of cells exhibiting a tumor stem cell phenotype and improving response to antitumor therapeutic modalities. Thus, by “treatment” or “treating” it is meant to inhibit the survival and/or growth of cells exhibiting a tumor stem cell phenotype, prevent the survival and/or growth of cells exhibiting a tumor stem cell phenotype, decrease the invasiveness of cells exhibiting a tumor stem cell phenotype, decrease the progression of cells exhibiting a tumor stem cell phenotype, decrease the metastases of cells exhibiting a tumor stem cell phenotype, increase the regression of cells exhibiting a tumor stem cell phenotype, and/or facilitate the killing of cells exhibiting a tumor stem cell phenotype. “Treatment” or “treating” is also meant to encompass maintenance of cells exhibiting a tumor stem cell phenotype in a dormant (or quiescent) state at their primary site as well as secondary sites. Further, by “treating or “treatment” it is meant to increase the efficacy as well as prevent or decrease resistance to antitumor therapeutics. “Treating” or “treatment” is also meant to encompass prolonged cancer remission, prevention of recurrence, decrease of cancer markers, reduction in cancer volume, reduction of pain, discomfort, and disability (morbidity), increase in quality of life associated with antitumor therapeutics, a decrease in mucositis, and a reduction in the need for anti-emetic agents and narcotic pain killers.

By “antitumor therapeutics” it is meant to include, but is not limited to, radiation therapies, thermal therapies, immunotherapies, hormone therapies, single agent chemotherapies, combination chemotherapies, chemo-irradiation therapies, adjuvant therapies, neo-adjuvant therapies, palliative therapies, and other therapies used by those of skill in the art in the treatment of cancer and other tumor malignancies.

By “increasing the efficacy”, it is meant to include an increase in potency and/or activity of the antitumor therapeutic and/or a decrease in the development of resistance to the antitumor therapeutic, and/or an increase in sensitivity of the tumor stem cells and/or tumor cells to the antitumor therapeutic.

By the phrase “inhibiting and preventing” as used herein, it is meant to reduce, reverse or alleviate, ameliorate, normalize, control or manage a biological condition. Thus, inhibiting and preventing a tumor stem cell phenotype in accordance with the present invention refers to preventing development, reversing or ameliorating development and/or normalizing, controlling or managing development of a tumor stem cell phenotype, cell, tumor and/or disease. Additionally inhibiting and preventing a malignant tumor in accordance with the present invention refers to preventing development, reversing or ameliorating development and/or normalizing, controlling or managing development of a malignant tumor. Similarly, inhibiting and preventing a malignant disease in accordance with the present invention refers to preventing development, reversing the ameliorating development and/or normalizing, controlling or managing development of a malignant disease.

Accordingly, administration of an agent to inhibit or prevent production or expression of nitric oxide synthase can be used both (1) prophylactically to inhibit and prevent a tumor stem cell phenotype, cell, tumor and/or disease from developing in animals at high risk for developing cancer or exposed to a factor known to increase nitric oxide synthase activity of cells, and (2) to treat cancer in animals by inhibiting metastases and development of resistance to antitumor therapeutics and increasing the efficacy of antitumor therapeutics.

As used herein, “tumor” is defined as an abnormal growth of tissue resulting from uncontrolled, progressive multiplication of cells and serving no physiological function; a neoplasm; and “neoplasm” is defined as an abnormal new growth of tissue that grows by cellular proliferation more rapidly than normal, continues to grow after the stimuli that initiated the new growth cease, shows partial or complete lack of structural organization and functional coordination with the normal tissue, and usually forms a distinct mass of tissue which may be either benign or malignant.

In accordance with these definitions, for purposes of the present invention, by “tumor stem cell phenotype” it is meant to encompass increases in metastasis, resistance to antitumor therapeutics, and angiogenesis. By “tumor stem cell phenotype, cell, tumor and/or disease” for purposes of the present invention, it is also meant to be inclusive of conditions in the spectrum leading to tumorigenic behavior and abnormal invasiveness such as hyperplasia, hypertrophy and dysplasia, as well as those cells and tissue that facilitate the malignant process. Examples of conditions in this spectrum include, but are not limited to, benign prostatic hyperplasia and molar pregnancy.

As evidenced by data presented herein, inhibition and prevention of a tumor stem cell phenotype in cells, tumors and/or diseases can be routinely determined by examining expression of genes including, but not limited to, nitric oxide synthase, Aldehyde Dehydrogenase-1 (ALDH-1) and Hoechst 33342; by examining cell invasiveness in in vitro or in vivo assays and/or by examining resistance of the cells to antitumor therapeutics. It is believed that elevated ALDH-lexpression and/or nitric oxide synthase activity may be observed in cells with a tumor stem cell phenotype. As will be understood by those of skill in the art upon reading this disclosure, however, other methods for determining gene expression via measurement of expressed protein or proteolytic fragments thereof can also be used.

For purposes of the present invention, by the term “nitric oxide antagonist” it is meant NO scavengers, or a functional equivalent thereof; any compound which accelerates the degradation of NO, inhibits the effects of NO, inhibits the production of NO synthase, generates or releases endogenous or exogenous NO inhibitors, including but not limited to, NG,NG-dimethylarginine (asymmetrical dimethylarginine, ADMA), NG-nitro-L-arginine methyl ester (L-NAME), aminoguanidine, nitro-L-arginine, N omega-nitro-L-arginine, N omega-monomethyl-L-arginine, 2-phenyl-4,4,5,5-tetramethylimidazoline-1-oxyl 3-oxide (PTIO), carboxy-PTIO, and carboxymethoxy-PTIO and any compound which in any other manner inhibits NO or a nitric oxide-like moiety or inhibits any stage of the NO production pathway; or any compound which inhibits or prevents NO utilization by the cell, when administered to an animal.

In another embodiment, anti-stem cell therapeutic agents used in the compositions and methods of the present invention can be inhibitors of hydroxyl radicals and/or superoxide anions (O₂). Hydroxyl radicals and superoxide anions are free radicals that can mediate toxic effects similar to NO, such as DNA fragmentation, cell damage and neuronal cell death. Free radical scavengers, such as any NO antagonist, superoxide dismutase (SOD) and small molecule superoxide dismutase mimetics and agonists can dramatically reduce the biological effects of NO and other free radicals. Examples of free radical scavengers can include, but are not limited to, curcumin, diacetylcurcumin, inhibitors of superoxide anions, epicatechin gallate, epigallocatechin gallate, gallocatechin, gallocatechin gallate, lipoic acid, tocopherol, hydroxytyrosol, ascorbic acid, balsalazide, caffeic acid, caffeic acid phenethyl ester, chlorogenic acid, chlorphyllin, delphinidin chloride, diosmin, ellagic acid, eugenaol, ferulic acid, fucoxanthin, gallic acid, ginkgolide B, herperidin, kaempferol, linoleic acid, luteolin, lycopene, N-acetyl-L-cysteine, oleic acid, resveratrol, rutin hydrate, se-(methyl)selenocysteine hydrochloride, seleno-L-methionine, sodium selenite, xanthophyll, carotene, courmaric acid, and salts and derivatives thereof.

As used herein “anti-NO therapy” refers to inhibition of nitric oxide, suppression of nitric oxide synthase, administration of nitric oxide antagonists, expression of superoxide dismutase (SOD) or superoxide dismutase complexes, administration of small molecule SODs, SOD mimetics, SOD analogs, SOD agonists and adminstration of free radical scavengers. Anti-NO therapy may be able to maintain the tumor cell under homeostatic stage, thus prevent the alteration of steroid receptor expressions, levels of expression, location, or alternatively, the ion-channels related to androgen and anti-androgen action etc. Thus, the administration of anti-NO therapy could keep patients under hormonal therapy for various forms of cancer under hormone responsive phase, preventing or delaying metastasis to secondary sites and transformation to more advanced, hormone-refractory/insensitive/insensitive cancers. Anti-NO therapy can be administered in conjunction with hormonal therapy during the treatment phase and/or can be used in the remission phase.

Anti-NO therapy can also be an antibody. As used herein, the term “antibody” refers to an immunoglobulin molecule capable of binding an epitope present on an antigen. The term is intended to encompasses not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required specificity. The antibody can be directed to the tumor cells, tumor stem cells, NO, NOS, inductible nitric oxide synthase (iNOS), aldehyde dehydrogenase (ALDH-1), glutathione S-transferase-pi (GST-7c), and apurinic/apyrimidinic endonuclease-1 (APE-1) or other applicable antigen.

In certain cases, anti-NO therapy can be used with chemo- and/or radio-therapeutic treatment to ensure eradication of cancers during the treatment phase, and can be used as stand-alone therapy during the remission. If low dose of chemotherapy is employed to prevent cancer recurrence, anti-NO therapy can be used with the low dose chemotherapy to prevent or prolong the time to cancer recurrence and eventually, prolong the survival time of cancer patients. The addition of anti-NO therapy could also 1) reduce the dependence on narcotic pain relief agents and thus associated adverse events due to their use and yet enhance the effectiveness of these agents, 2) prevent progressive lost of bone mineral density and thus, the development of osteoporosis and the risk of bone fracture, and 3) improve overall quality of life of cancer suffers.

Screening for Anti-NO Therapeutics

In certain embodiments, the present invention concerns a method for identifying, prioritizing and testing compounds that will be of a potential therapeutic value. It is disclosed that this screening technique will prove useful in the general prioritization and identification of compounds that will serve as lead therapeutic compounds for drug development. The invention will be a useful addition to laboratory analyses directed at identifying new and useful compounds for the intervention of a variety of diseases and disorders including, but not limited to, Alzheimer's disease, other disorders and diseases of the central nervous system, metabolic disorders and diseases, cancers, diabetes, depression, immunodeficiency diseases and disorders, immunological diseases and disorders, autoimmune diseases and disorders, gastrointestinal diseases and disorders, cardiovascular diseases and disorders, inflammatory diseases and disorders, and infectious diseases, such as a microbial, viral or fungal infections

In one embodiment, a method for determining the cytotoxicity of candidate substances to tumor stem cells is disclosed. The method can employ a method including generally: a) culturing HNO stem cells in culture medium that comprises a plurality of concentrations of said chemical compound; b) measuring a first indicator of cell health at one or more concentrations of said chemical compound; c) measuring a second indicator of cell health at one or more concentrations of said chemical compound; d) measuring a third indicator of cell health at one or more concentrations of said chemical compound; and e) predicting a toxic concentration.

As disclosed herein, the term “health indicator” refers to characteristics of the cell after, during or prior to treatment with a nitric oxide therapy, such as, but not limited to, reduced or altered cell viability, altered cell surface marker expression, reduced tumorigenicity, stem cell ratio to tumor cells, UV sensitivity, radiation sensitivity, temperature sensitivity, protein expression (e.g. aldehyde dehydrogenase (ALDH-1), glutathione S-transferase-pi (GST-π), inductible nitric oxide synthase (iNOS) and apurinic/apyrimidinic endonuclease-1 (APE-1)), and DNA degradation (e.g. COMET assays).

Viability of the HNO stem cells can be assessed before, after, during exposure to an anti-NO therapy by any of methods known or frequently used in the art. Such methods can include chemical analysis, flow cytometry, trypan blue exclusion, ELISA, measuring metabolic activity, localization of proteins, localization of nucleic acids, measuring protein content and measuring nucleic acid content, membrane characteristics and other known assays used frequently in the art. Common assays used can include 3-(4,5-dimethylthiazol-2-yl)-2,5-diphenyltetrazolium bromide assays, Hoechst staining (33342 and 33258) of nucleic acids, propidium idodide staining of nucleic acids, measuring ATP contained in the cells, resazurin and formazan assays, methyl violet, TUNEL assays, flow cytometry of molecules found in viable or non-viable cells, such as mitochondrial surface markers, nucleic acids, and other known molecules and assays used to differentiate viable from non-viable cells.

Another aspect can include determining cell surface marker expression of the HNO cells exposed to anti-NO therapy. Surface makers can include, but are not limited to, CD24, CD34, CD38, CD44, CD117, CD133, CD166 and others that may be associated with a particular cell type. For example, hematopoietic cells express CD45, lung cells express ALDH-1, epithelial cells express CD50 and CD44, etc.

Tumorigenicity can also be determined from the cells. Common methods can include in vitro assays, such as contact inhibition, serum free growth, migration assays, angiogenesis, etc. Reduced tumorigenicity can be characterized by contact inhibition, lack of or reduced serum free growth, inability to or reduced migration in migration assays, reduced or lack of angiogenesis, etc. Common methods can also include in vivo assays, such as tumor formation in animal models. In vivo growth of HNO cells treated with anti-NO therapy can include intravenous, intraperitoneal or subcutaneous injection of cells. The injections can include limiting dilutions of the tumor cells to determine efficacy of the anti-NO therapy in comparison to non-treated tumor cells.

One aspect can include determining temperature sensitivity or resistance of the HNO cells prior to, after or during exposure to an anti-NO therapy. Temperature sensitivity or resistance can be measured in cells seeded in, for example, 96 well plates, 384 well plates, 1536 well plates or other sizes. For high throughput screening of temperature sensitivity or resistance a PCR thermocycler can be used. A step gradient from 15° C. to 60° C. in degree change/min increments can be used. For example, 5° C. per minute increments can be used. In other embodiments, about a 1° C., 2° C., 3° C., 4° C., 5° C., 6° C., 7° C., 8° C., 9° C. and 10° C. increments can be used. The temperature change can about every 20 sec, 30 sec, 40 sec, 50 sec, 1 min, 2 min, 3min, 4 min, 5 min, 6 min, 7 min, 8 min, 9 min and 10 mins.

One aspect can include determining radiation sensitivity or resistance of the HNO cells prior to, after or during exposure to an anti-NO therapy. The cells can be exposed to varying doses of radiation. Radiation sensitivity or resistance can be measured in cells seeded in, for example, 96 well plates, 384 well plates, 1536 well plates or other sizes. The cells can be irradiated with 1 Gy, 2 Gy, 3 Gy, 4 Gy, 5 Gy, 6 Gy, 7 Gy, 8 Gy, 9 Gy, 10 Gy, 11 Gy, 12 Gy, 13 Gy, 14 Gy, 15 Gy, 16 Gy, 17 Gy, 18 Gy, 19 Gy, 20 Gy, 21 Gy, 22 Gy, 23 Gy, 24 Gy, 25 Gy, 26 Gy, 27 Gy, 28 Gy, 29 Gy, and 30 Gy. The cells can be irradiated with split doses or in a single dose.

Another aspect can include determining UV sensitivity or resistance of the HNO cells prior to, after or during exposure to an anti-NO therapy. The cells can be exposed to varying doses of UV radiation. UV sensitivity or resistance can be measured in cells seeded in, for example, 96 well plates, 384 well plates, 1536 well plates or other sizes. The cells can be exposed to UV lights of varying intensities, for example 254 nm, 13.4 W ultraviolet output with the cells in a plate positioned ˜51 cm from light source. In one exemplary embodiment, the cells can be irradiated with the UV light source for approximately 0 sec, 30 sec, 1 min, 2 min, 3 min, 4 min, 5 min, 6 min, 7 min, 8 min, 9min or 10 min.

Another aspect can include determining the ratio of HNO stem cells to tumor cells. Using any of the assays above using limiting dilutions of the cells to determine the number of self-renewing stem cells can be used to estimate the ratio of stem cells to tumor cells.

The methods may also determine, monitor or otherwise predict cytotoxicity in a variety of HNO tumor stem cell tissue types. It should be understood that the HNO tumor stem cells can be derived from a single or a multiplicity of sites within an organism. The cellular toxicity associated with those sites may also be monitored with application of the particular test compound as well as sites remote or surrounding the natural site where the HNO tumor stem cells are derived. Therefore, parental cell lines may be used in assays of cytotoxicity as well as primary tissues and other cell lines.

In another embodiment, the present invention may include performing such methods with more than one HNO stem cell type. For example, to analyze the toxicity of an anti-tumor compound, it would be beneficial to examine the effects of the compound on different cell types, i.e., HNO stem cells derived from tumor cells, HNO stem cells derived from normal tissue, parental cells, primary tissue, proliferating cells derived from normal tissue and non-proliferating cells derived from normal tissue. The use of these different cell types allows for the differentiation between target versus off target effects of the anti-tumor compound. Alternatively, the same cell type from two or more different mammalian species may be utilized in accordance with the present invention. The use of cells from different species allows for the identification of potential species specific toxicity of a compound.

In some embodiments, the cells can be seeded in multiwell (e.g., 96-well) plates and allowed to reach log phase growth. In HNO stem cells, this growth period is approximately 2-24 hours. Preferred media and cell culture conditions for multiple HNO stem cell lines are detailed in the Examples.

Once the cell cultures are thus established, various concentrations of the compound being tested are added to the media and the cells are allowed to grow exposed to the various concentrations for 24 hours. While the 24 hour exposure period is described, it should be noted that this is merely an exemplary time of exposure and testing the specific compounds for longer or shorter periods of time is disclosed to be within the scope of the invention. As such it is disclosed that the cells may be exposed for 6, 12, 24, 36, 48 or more hours. Increased culture times may sometimes reveal additional cytotoxicity information, at the cost of slowing down the screening process.

Furthermore, the cells may be exposed to the test compound at any given phase in the growth cycle. For example, in some embodiments, it may be desirable to contact the cells with the compound at the same time as a new cell culture is initiated. Alternatively, it may be desirable to add the compound when the cells have reached confluent growth or arc in log growth phase. Determining the particular growth phase cells are in is achieved through methods well known to those of skill in the art.

The varying concentrations of the given test compound are selected with the goal of including some concentrations at which no toxic effect is observed and also at least two or more higher concentrations at which a toxic effect is observed or one or more indicators of HNO cell health is altered. A further consideration is to run the assays at concentrations of a compound that can be achieved in vivo. For example, assaying several concentrations within the range from 0 micromolar to about 300 micromolar is commonly useful to achieve these goals. It will be possible or even desirable to conduct certain of these assays at concentrations higher than 300 micromolar, such as, for example, 350 micromolar, 400 micromolar, 450 micromolar, 500 micromolar, 600 micromolar, 700 micromolar, 800 micromolar, 900 micromolar, or even at millimolar concentrations. The estimated therapeutically effective concentration of a compound provides initial guidance as to upper ranges of concentrations to test. Additionally, other assays to analyze a range of concentrations can include at least two concentrations at which cytotoxicity is observable in an assay. It has been found that assaying a range of concentrations as high as 300 micromolar often satisfies this criterion.

In an exemplary set of assays, the test compound concentration range can comprise dosing concentrations which yield final growth media concentration of 0.05 micromolar, 0.1 micromolar, 1.0 micromolar, 5.0 micromolar, 10.0 micromolar, 20.0 micromolar, 50.0 micromolar, 100 micromolar, and 300 micromolar of the compound in culture media. As mentioned, these are exemplary ranges, and it is envisioned that any given assay will be run in at least two different concentrations, and the concentration dosing may comprise, for example, 4, 5, 6, 7, 8, 9, 10, 11, 12, 13, 14, 15 or more concentrations of the compound being tested. Such concentrations may yield, for example, a media concentration of 0.05 micromolar, 0.1 micromolar, 0.5 micromolar, 1.0 micromolar, 2.0 micromolar, 3.0 micromolar, 4.0 micromolar, 5.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 55.0 micromolar, 60.0 micromolar, 65.0 micromolar, 70.0 micromolar, 75.0 micromolar, 80.0 micromolar, 85.0 micromolar, 90.0 micromolar, 95.0 micromolar, 80.0 micromolar, 110.0 micromolar, 120.0 micromolar, 130.0 micromolar, 140.0 micromolar, 150.0 micromolar, 160.0 micromolar, 170.0 micromolar, 180.0 micromolar, 190.0 micromolar, 200.0 micromolar, 210.0 micromolar, 220.0 micromolar, 230.0 micromolar, 240.0 micromolar, 250.0 micromolar, 260.0 micromolar, 270.0 micromolar, 280.0 micromolar, 290.0 micromolar, and 300 micromolar in culture media. It will be apparent that a cost-benefit balancing exists in which the testing of more concentrations over the desired range provides additional information, but at additional cost, due to the increased number of cell cultures, assay reagents, and time required. In one embodiment, ten different concentrations over the range of 0 micromolar to 300 micromolar are screened.

Typically, the various assays described in the present specification can employ cells seeded in, for example, 96 well plates, 384 well plates, 1536 well plates or other sizes. The cells can then be exposed to the test compounds over a concentration range, for example, 0-300 micromolar. The cells can be incubated in these concentrations for a given period of, for example, 6 and/or 24 hours. Subsequent to the incubation, the multiple assays can be performed for each test compound. In one embodiment, all the assays are performed at the same time such that a complete set of data are generated under similar conditions of culture, time and handling. However, it may be that the assays are performed in batches within a few days of each other.

In specific embodiments, the indicators of cell health and viability include but are not limited to, indicators of cellular replication, mitochondrial function, energy balance, membrane integrity and cell mortality. In other embodiments, the indicators of cell health and viability further include indicators of oxidative stress, metabolic activation, metabolic stability, enzyme induction, enzyme inhibition, and interaction with cell membrane transporters.

The compounds to be tested may include fragments or parts of naturally-occurring compounds or may be derived from previously known compounds through a rational drug design scheme. It is proposed that compounds isolated from natural sources, such as animals, bacteria, fungi, plant sources, including leaves and bark, and marine samples may be assayed as candidates for the presence of potentially useful pharmaceutical compounds. Alternatively, pharmaceutical compounds to be screened for toxicity could also be synthesized (i.e., man-made compounds).

Once all data for a given cluster of assays are received, the data can be analyzed to obtain a detailed profile of the compound's toxicity. For example, the data are collated over a dose response range on a single graph. In such an embodiment, the measurement evaluated for each parameter (i.e., each indicator of cell health) at any given concentration can be plotted as a percentage of a control measurement obtained in the absence of the compound. However, it should be noted that the data need not be plotted on a single graph, so long as all the parameters can be analyzed collectively to yield detailed information of the effects of the concentration of the compound on the different parameters to yield an overall toxicity profile. As set forth below, this overall toxicity profile can facilitate a determination of a plasma concentration that is predicted to be toxic in vivo. This plasma concentration represents an estimate of the sustained plasma concentration in vivo that would result in toxicity, such as HNO tumor stem cell toxicity.

Pharmaceutical Compositions

As used herein, the anti-NO therapy can be administered with a pharmaceutically acceptable carrier. The term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 describes a variety of different carriers that are used in formulating pharmaceutical compositions and known techniques for the preparation thereof. Some examples of materials that are pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The active agents of the invention are preferably formulated in dosage unit form for ease of administration and uniformity of dosage. The expression “dosage unit form” as used herein refers to a physically discrete unit of active agent appropriate for the cells or patient to be treated. It will be understood, however, that the total daily usage of the compositions of the present invention are decided by an attending physician, within the scope of sound medical judgment. For any active agent, the therapeutically effective dose can be estimated initially either in cell culture assays or in animal models, usually mice, rabbits, dogs, or pigs. The animal model is also used to achieve a desirable concentration range and route of administration. While direct application to the cell is envisioned as the route of administration, in vivo or ex vivo, such information can then be used to determine useful doses and additional routes for administration in animals or humans.

The term “subject” as used herein refers to any living organism, including, but not limited to, humans, nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like. The term does not denote a particular age or sex. In a specific embodiment, the subject is human.

The terms “treating,” “treatment” or “intervention” refer to the administration of one or more therapeutic agents or procedures to a subject who has a condition or disorder or a predisposition toward a condition or disorder, with the purpose to prevent, alleviate, relieve, alter, remedy, ameliorate, improve, affect, slow or stop the progression, slow or stop the worsening of the disease, at least one symptom of condition or disorder, or the predisposition toward the condition or disorder.

A therapeutically effective dose refers to that amount of active agent that ameliorates the symptoms or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use

### EXAMPLES

### Example 1

### Materials and Methods

Tissue Culture: The cell lines used were an adenocarcinoma cell line (A549), five human squamous head & neck cell lines (SCC016, SCC040, SCC056, SCC114, SCC116), four breast adenocarcinomas (T-47D, Hs578t, BT-20, and MCF-7), four human colon cell lines, and three prostate cell lines. In addition a normal human cell line WI-38 and mouse cell lines (PO7 and NIH-3T3 cells) were adapted to produce stem cell cultures. Growth of the cultures was maintained at 37° C., 95% air/5% CO2 in 100% humidity or the growth conditions known for the particular cell line.

All media and supplements were purchased from Invitrogen Corporation (Carlsbad, Calif., USA) except for those that are noted. Five human head and neck squamous cell carcinoma (HNSCC) cell lines (three originating from the tongue: SCC016, SCC040, and SCC056; one from the floor of mouth: SCC114; and one from the alveolar ridge: SCC116) were grown in MEM media. All media were supplemented with 10% fetal calf serum (FCS) inactivated at 56° C. for 30 min, 100 U/mL of penicillin, 100 μg/mL of streptomycin, 2 mM L-glutamine, and 2.5 μg/mL of Amphotericin B solution. The MEM media were additionally supplemented with 100 mM MEM nonessential amino acids and 1 mM sodium pyruvate (Mediatech, Inc., Manassas, Va., USA). Cell lines were grown in a humidified incubator at 37° C. and 5% CO2. The nitric oxide donor (Z)-1-[2-(2-aminoethyl)-N-(2-ammonioethyl) amino]diazen-1-ium-1,2-diolate (DETA-NONOate) (Sigma-Aldrich Corp., St. Louis, Mo., USA) was utilized in this study. DETA-NONOate stock solutions were prepared in sterilized water and sterile filtered (0.22 μm). Solutions were aliquoted and stored at −20° C. until ready for use.

Long Term NO Adapted Cultures: Tumor cells were adapted to grow in increasingly higher levels of NO. The cells lines were adapted to increasing levels of NO by adding new NO donor every 3-4 days to the cultures. The adaptation process was initiated by passaging cells with trypsin-EDTA and transferring the cells to a new flask containing media supplemented with 50 μM DETA-NONOate. The DETANONOate solution was prepared immediately prior to the addition of cells into the growth media. Cells were then incubated at 37° C. and 5% CO2 until they reached ˜90% confluency.

When the cells had grown enough to be split, one flask remained at the current NO concentration, and another was subjected to a 50 uM increase of DETA-NONOate. Some cell death was observed when some of the tumor cell lines were exposed to the next higher dose of DETA-NONOate, but the cultures eventually recovered and grew robustly. These preliminary experiments were repeated three times. The high NO (HNO) cultures were maintained long term with 600 uM DETA-NONOate added every 3 to 4 days.

In addition to the adapted cells, separate “parent” cells were maintained as controls. No nitric oxide donor was added to the parent cells, and these cells were grown under standard conditions. The cells were replenished with media containing DETANONOate every 2-3 days, both during the adaptation process and the subsequent maintenance of the fully adapted cells. Media were also replenished every 2-3 days for the parent cells.

Viability of Long Term NO Adapted Cultures: Cells from each of the cell lines were seeded (100 μL) into 96-wellmicrotiter plates and grown for 24 h, to ˜70% confluency. The media was then removed, and the cells were treated with 100-μL media containing varying concentrations (0-600 μM) of DETA-NONOate. Following an additional 72-h incubation period, cell proliferation/viability was assessed using the 3-(4,5-dimethylthiazol-2-yl)-2,5-diphenyltetrazolium bromide (MTT) assay. Media was removed from each well, and 100 μL of 2 mg/mL of MTT (Sigma-Aldrich Corp., St. Louis, Mo., USA) in phosphate buffer saline (PBS) was added. Plates were then placed in an incubator (37° C.) for 5 h, during which time purple formazan crystals formed upon the reaction of the MTT with mitochondrial dehydrogenases of viable cells. Following the incubation period, the MTT solution was removed, and 100 μL of dimethyl sulfoxide (DMSO) was added. The absorbance of the resulting purple solution in each well was read (540 nm) using a SpectraMax® Plus 384 spectrophotometer (Molecular Devices, Inc., Sunnyvale, Calif., USA). Each experiment was independently conducted a minimum of three times, and a minimum of four replicate wells were tested for each cell line at each concentration. Data were normalized to the mean optical density of the untreated control cells.

Parent A549, a human lung adenocarcinoma cell line, and A549-HNO cells were seeded (100 μL) into 96-well plates in the appropriate media (i.e., standard media for parent cell lines; media treated with 600 μM DETA-NONOate for the adapted cells). The plates were incubated for 24 hours, after which time the media was replaced with analogous serum-less media (i.e., lacking the 10% FCS). With the exception of the FCS, all other components of the media, including the additional DETA-NONOate for the HNO adapted cells, were identical to the original formulation used. Plates were then incubated for an additional 24, 48, and 72 hours. MTT assays were performed at each time point, as described above.

Growth On Soft Agar: Growing cells on soft agar was a high throughput 96 well assay in which 96 well plates are coated with soft agar. A soft agar mixture consisting of 50% low-melting point agarose (Sigma A-9539, Sigma-Aldrich Corp., St. Louis, Mo., USA) and 50% 2× MEM supplemented with 20% FCS, 200 U/mL of penicillin, 200 m/mL of streptomycin, 4 mMLglutamine, and 5 μg/mL of Amphotericin B solution was prepared. This mixture was incubated at 42° C. for at least 30 min, after which time the agarose mixture (100 μL) was loaded into a 96-well microtiter plate (100 μL). Concurrently, cells were grown, harvested, and counted (or FACS sorted). The agarose was then allowed to harden overnight at 4° C., after which time parent and HNO-adapted cells (100 μL) were added on top of the hardened agar. (HNO-adapted cells were added in standard 1× growth media supplemented with 600 μM DETANONOate; parent cells were added in standard 1× growth media lacking additional DETA-NONOate.) In order to allow the cells to attach to the soft agar, plates were incubated for 12 h at 37° C.; after this time, the media above the agar were removed. The plates were incubated for another 24 or 72 h, at which time MTT proliferation/viability assays were carried out. A 2 mg/mL ofMTT in PBS solution (100 μL) was added to each well (on top of the agar), and the plates were incubated at 37° C. for 5 h. During this time, purple formazan crystals developed. Following the incubation period, theMTTsolution was removed, and the remaining crystals were dissolved in DMSO (100 μL), yielding a purple solution. The resulting supernatant in each well was then transferred to a new 96-well microtiter plate. This new plate was used to obtain the absorbance reading for each well as described above. Four replicate wells were tested for each cell line at each time point. Data were normalized to the mean optical density value at the 24 h time point. Data were plotted as the mean normalized absorbance±standard deviation.

FACS Analysis: To determine viability of cells, and ascertain the percentage of cells undergoing apoptosis, Hoechst 33342 dye (excitation/emission maxima ˜350/461 nm, when bound to DNA), which stains the condensed chromatin of apoptotic cells more brightly than the chromatin of normal cells, was used. Cells (2-4×10⁶/ml) were stained in media containing 0.1 to 1 mM Hoechst 33342 dye less than one hour before FACS sorting or analysis. Antibodies to GST-π, ALDH-1, CD-24, CD-34, CD-38, CD-44, CD-133, CD-166, were obtained from commercial suppliers. In brief, cells, either previously stained with Hoechst 33342 or unstained, (parent cell lines, HNO adapted cell lines, FACS sorted cells) were immunostained with one or more of the antibodies. Cells were stained with 85 μL of antibody at a 1:20 dilution in 1.7 mL of 1% BSA/PBS mixture and incubated in 80 μL per slide for 45 minutes. Cells were counterstained with 30 μL of BAB secondary antibody in 6 mL of 1% BSA/PBS mixture, 60 μL of each ABC secondary antibody solution in 6 mL of PBS or 0.3 g of DAB secondary antibody in 600 mL of PBS with 300 μL of H₂O₂. Positive and negative controls were run in each experiment, along with select duplicate slides. The slides were coded and independently read twice for staining intensities. A double label immunostaining method was used if needed, as well as CCD computer assisted image analysis. Attached is the chart of the CD markers that have been found on some of the HNO cell lines that we have adapted and tested to date. Other cells (normal and tumor) would have different CD and non-CD markers expressed on their cell surfaces.

FACS Cell Cycle Analysis: To prepare samples for fluorescence-activated cell sorting analysis, parent and HNO cells were harvested and fixed in ice-cold 70% ethanol. The cell suspension was washed with PBS twice and treated with 50 μL of 100 μg/mL ribonuclease (Sigma-Aldrich Corp., St. Louis, Mo., USA) for 5 min. A 50 μg/mL solution of propidium iodide (200 μL) was then added directly to the cells. An EPICS Elite ESP flow cytometer/cell sorter (Beckman Coulter Inc., Fullerton, Calif., USA) was used to determine the cell cycle profile for each cell line. Excitation was achieved using a 15 mW air-cooled 488 nm argon ion laser; propidium iodide emission (λmax=620 nm) was measured using a 610-nm long-pass filter. For each cell line, the percentage of cells in each phase of the cell cycle was reported for the experiment.

Exposure to Radicals: Cells were exposed to varying concentrations of hydrogen peroxide to determine if the adapted cells were resistant to high free radical environments generated by oxygen-based free radicals. Parent and adapted cells were seeded (100 μL) into 96-well microtiter plates in the appropriate media (parent cells in media without NO donor, HNO cells in media supplemented with 600 μM DETA-NONOate) and incubated overnight, reaching approximately 70% confluency by the following day. Cells were treated with varying concentrations (0-1.78 mM) of hydrogen peroxide (30% w/w solution, Sigma H1009, Sigma-Aldrich Corp., St. Louis, Mo., USA) and incubated at 37° C. for an additional 24 h. MTT cell viability/proliferation assays were then performed, as described above. A minimum of four replicate wells were measured for each cell line at each concentration, and assays were repeated in triplicate. Data were normalized against the mean of the untreated control cells, and values lying outside of two standard deviations were removed. Data were plotted as the mean normalized absorbance±standard error.

Radiation Resistance: Parent and HNO cells were exposed to varying doses of radiation via an intensity-modulated radiation therapy (IMRT) treatment plan. Cells were seeded into 96-well microtiter plates (100 μL) in the appropriate media (i.e., standard MEM media for parent cells; MEM media supplemented with 600 μM DETA-NONOate for HNO cells). Irradiation was carried out when the cells reached approximately ˜50% confluency. Approximately 3 h prior to receiving the radiation, an additional 150 μL of media was added to each well, resulting in a total volume of 250 μL per well.

The microtiter plates were loaded into a previously developed head and neck phantom, and a computed tomography (CT) scan (PQ5000, Philips Medical Systems Inc., Andover, Mass., USA) of the phantom was acquired. An Eclipse treatment planning system (Varian Corp., Palo Alto, Calif., USA) was used to develop an IMRT treatment plan consisting of two 0235 cm3 cuboidal planning target volumes (PTVs). Each PTV resided at opposite ends of the plates, and each contained four rows of eight wells (four rows of parent cells and four rows of HNO cells). Six equally separated (52°) beam angles were used, ranging from 28° to 336°. After being placed on the patient treatment table, the phantom was irradiated at 1, 2, 5, 10, 14, or 28 Gy using 6 MV photons delivered by a clinical linear accelerator (2100CD, Varian Corp., Palo Alto, Calif., USA). A fixed 2:1 ratio was used between the two PTVs, and each plate received two doses (1 and 2 Gy, 5 and 10 Gy, or 14 and 28 Gy), delivered as uniformly as possible. The experiment, including transport of plates to and from the IMRT facility, took approximately 60 min. A separate plate that was transported to the IMRT facility, but not exposed to radiation, served as the control. Following irradiation, the plates were incubated for an additional 96 h at 37° C.

The diphenylamine (DPA) assay [16], which colorimetrically measures the amount of DNA in cells, was used to determine cell viability. All reagents for the DPA assay were purchased from Sigma-Aldrich Corporation (St. Louis, Mo., USA). Following the 96 h incubation, the media was aspirated from the cells. A 1:5 mixture (60 μL) of chilled acetaldehyde (0.16% in water) and perchloric acid (20% v/v) was added, followed by a 4% DPA solution in glacial acetic acid (100 μL). Following another 24 h incubation period at 37° C., the absorbance of each well was read at 595 nm using a SpectraMax® Plus 384 spectrophotometer (Molecular Devices, Inc., Sunnyvale, Calif., USA). For each dose tested, eight replicate microtiter wells were tested, and three independent trials were carried out for each cell line. Data was normalized against the mean absorbance readings of the untreated control cells, and data points lying outside of two standard deviations were removed. Values were reported as the mean normalized absorbance±standard error.

UV Resistance: Parent and HNO cells were seeded (50 μL) into 96-well microtiter plates in the appropriate media (i.e., standard MEM media for parent cells; MEM media supplemented with 600 μM DETA-NONOate for HNO cells) and established overnight to ˜70% confluency. In a sterile hood, the cells were exposed to a UV germicidal light (254 nm, 13.4 W ultraviolet output; plate positioned ˜51 cm from light source) for 0, 2, 4, 6, 8, or 10 min. To ensure direct irradiation of the cells, the lid of the microtiter plate was removed during radiation. After UV exposure, an additional 100 μL of fresh media was added to each well (for a total volume of 150 μL/well), and the plates were incubated at 37° C. for an additional 72 h.

Cell proliferation/viability was then measured using the 3-(4,5-dimethylthiazol-2-yl)-2,5-diphenyltetrazolium (MTT) assay. To carry out the assay, media was removed from the wells, and 100 μL of 2 mg/mL MTT (Sigma-Aldrich Corp., St. Louis, Mo., USA) in phosphate buffer saline (PBS) was added. After a 5-h incubation period (at 37° C.), the MTT was removed from the cells, leaving purple formazan crystals. The crystals were dissolved in 100 μL of DMSO, and the absorbance of each well was read at 540 nm using a SpectraMax® Plus 384 spectrophotometer (Molecular Devices, Inc., Sunnyvale, Calif., USA). For each experimental condition, eight replicate wells were setup per cell line, and a minimum of three independent trials were carried out (n=3 for SCC040 parent/HNO and SCC056 parent/HNO; n=4 for SCC016 parent/HNO). Data was normalized against the mean absorbance readings of the untreated control cells, and data points lying outside of two standard deviations were removed. Values were reported as the mean normalized absorbance±standard error.

Cell proliferation/viability was then measured using the 3-(4,5-dimethylthiazol-2-yl)-2,5-diphenyltetrazolium (MTT) assay. To carry out the assay, media was removed from the wells, and 100 μL of 2 mg/mL MTT (Sigma-Aldrich Corp., St. Louis, Mo., USA) in phosphate buffer saline (PBS) was added. After a 5-h incubation period (at 37° C.), the MTT was removed from the cells, leaving purple formazan crystals. The crystals were dissolved in 100 μL of DMSO, and the absorbance of each well was read at 540 nm using a SpectraMax® Plus 384 spectrophotometer (Molecular Devices, Inc., Sunnyvale, Calif., USA). For each experimental condition, eight replicate wells were setup per cell line, and a minimum of three independent trials were carried out (n=3 for SCC040 parent/HNO and SCC056 parent/HNO; n=4 for SCC016 parent/HNO). Data was normalized against the mean absorbance readings of the untreated control cells, and data points lying outside of two standard deviations were removed. Values were reported as the mean normalized absorbance±standard error.

CytoSelect Assay: In brief, cells were suspended in media without serum. Then the cells were plated in the upper chamber of a migration plate separated from the lower chamber with a polycarbonate membrane having a pore size of 8 μm or 3 μm. The lower chamber contained media with fetal calf serum. Cells were then attracted to the fetal calf serum components and were allowed to migrate from the upper chamber to the lower chamber. Cells were detached from the lower side of the membrane, and detected using CyQuant GR dye provided by the manufacturer at 480/538 nm in a 96 well fluorometer.

COMET Assays: COMET assays are a molecular method to determine the amount of DNA fragmentation found in a single cell. A CometAssay™ kit (Trevigen, Inc., Gaithersburg, Md., USA) was used to measure ongoing DNA damage of the parent and HNO cell lines. Cells were grown under the previously described conditions (i.e., standard MEM media for parent cells; MEM media supplemented with 600 μM DETA-NONOate for HNO cells). The cells were harvested, counted using a hemocytometer, adjusted to a total concentration of 1×105 cells/mL, and resuspended in PBS. Immediately thereafter, a 10-μL aliquot of the cell suspension was mixed with 100 μL of 1% low melting point agarose (Sigma-Aldrich Corp., St. Louis, Mo., USA) at 37° C.; this solution was then quickly dispensed (75 μL) onto a CometSlide™. The slide was immersed in cold lysing buffer (2.5 mM NaCl, 100 mM Na2EDTA, 10 mM Tris, 1% N-lauryl sarcosine sodium salt, pH 10) and kept at 4° C. for 1 h. The slide was then rinsed gently with Tris-borate buffer (TBE; 10.8 g Tris, 5.5 g boric acid, and 0.93 g Na2EDTA in 1 L dH2O) twice and placed on a horizontal gel electrophoresis platform covered in TBE buffer. Slides were run at 13 V for 10 min, then dipped into 70% ethanol and air-dried. Each slide was fixed and stained with 100 μL of silver staining solution and cover-slipped. Comet tails were imaged on a Reichert Microstar IV microscope (Reichert, Inc., Depew, N.Y., USA) at 400×; images were captured using the Dazzle Multimedia software package (Pinnacle Systems, Inc., Mountain View, Calif., USA). Tail lengths were measured in pixel units, as the distance from the center of the cell nucleus to the tip of the tail. For each slide, a minimum of 15 Comet tails were measured (n=15 for SCC016 parent/HNO, SCC040 parent/HNO; n=20 for SCC056 parent/HNO). Values are reported as the mean Comet tail length±standard error.

Immunoblotting: Western blots were performed to detect the expression of iNOS, eNOS, and apurinic/apyrimidinic endonuclease-1 (APE1) in parent and HNO cells of SCC016, SCC040, and SCC056. Cells were lysed with 1× SDS sample buffer (62.5 mM Tris-HCl, pH 6.8, 2% SDS, 10% glycerol, 50 mM DTT, and 0.01% bromophenol blue). Proteins were separated on SDS-PAGE and transferred to nitrocellulose membrane. Nonspecific binding was blocked with 5% nonfat dry milk in TBST (10 mM Tris-HCl, pH 7.5, 0.15 M NaCl, and 0.05% Tween 20) overnight at 4° C. and then incubated with anti-iNOS antibody (Abcam Inc., Cambridge, Mass., USA; Catalog #ab3523), anti-eNOS antibody (Cell Signaling Technology, Inc., Danvers, Mass.; Catalog#9572), anti-GST-pi antibody (Thermo Fisher Scientific Inc., Waltham, Mass., USA; Catalog #RB-050-A1), or anti-APE1 antibody (Abcam Inc., Cambridge, Mass., USA; Catalog #ab194-50) in TBST containing 5% nonfat dry milk for 2 h at 22° C. After being washed with TBST, the membranes were incubated with horseradish peroxidase-conjugated goat anti-mouse secondary antibody or with horseradish peroxidase-conjugated goat anti-rabbit secondary antibody for 1 h. The membranes were washed, and the protein bands were detected by enhanced chemiluminescent substrate (Thermo Fisher Scientific Inc., Waltham, Mass., USA, SuperSignal West Pico Chemiluminescent Substrate). Protein band densities were quantified in Adobe Photoshop V7.0 (Adobe Systems Inc., San Jose, Calif., USA) following a previously reported method. Quantitative analysis of the data is presented as the average of triplicate measurements±standard error. Error bars represent variations of less than 5%. In cases where multiple bands were observed for an antibody in a given cell line, the densities of the individual bands were summed together. The relative expression level was normalized to β-actin, which was used as a loading control.

Immunohistochemical Analysis: Well-characterized, commercially available monoclonal antibodies against eNOS, iNOS, GST-pi, and APE1, were used for immunoperoxidase analysis. The immunohistochemical method was chosen to evaluate the intracellular localization in addition to the intensity of the expression. Tissue specimens were formalin-fixed and paraffin-embedded. A single paraffin block was selected for each specimen, from which 7 μm sections were cut. A series of xylene, graded alcohol, and water immersion steps were carried out to de-paraffinize the individual sections. Slides were then treated with 3% hydrogen peroxide to neutralize endogenous peroxidase activity, after which they were subjected to normal horse serum in 1% bovine serum albumin in phosphate-buffered saline. Immunostaining was carried out with the appropriate antibody (eNOS, iNOS, GST-pi, APE lor no primary antibody) and the avidin-biotin complex detection method. All incubation steps were carried out for 20 min. The chromogen used was 3,3′-diaminobenzidine tetrahydro-chloride. Finally, slides were counterstained with Harris' hematoxylin for 1 min, dehydrated, and cover-slipped.

A blinded histopathological review was performed on all slides. For each specimen, the percentage of positive cells was determined and the staining intensity was graded on a scale of 0 to 3+, with 0 indicating no staining, 1+ weak, 2+ moderate, and 3+ strong staining.

Antibodies to GST-π, ALDH-1, CD-24, CD-34, CD-38, CD-44, CD-133, and CD-166 obtained from commercial suppliers, were also used for immunostaining. In brief, cytospins were made of the cells under study (parent cell lines, HNO adapted cell lines, FACS sorted cells) and immunostained with antibodies. Positive and negative controls were run in each experiment, along with select duplicate slides. The slides were coded and independently read twice for staining intensities. A double label immunostaining method was used if needed, as well as CCD computer assisted image analysis. Attached is the chart of the CD markers that have been found on some of the HNO cell lines that we have adapted and tested to date. Other cells (normal and tumor) would have different CD and non-CD markers expressed on their cell surfaces.

GeneChip Array: Expression profiles were generated from A549 parent cells compared to HNO A549a cells. Briefly, total RNA was extracted from duplicate samples of A549 parent cells and HNO A549a cells using the RNA extraction kits. The amount and quality of RNA was assessed by UV spectrophotometer. cRNA was generated using standard T7 amplification protocol. Second-strand cDNA synthesis was done in the presence of DNA Polymerase I, DNA ligase, and RNase H, and the resulting double-stranded cDNA was blunt-ended using T4 DNA polymerase and purified by phenol/chloroform extraction. No second-round amplification was done. cRNA was generated in the presence of biotin-ribonucleotides using an RNA transcript labeling kit. The biotin-labeled cRNA was purified using Qiagen RNeasy columns (Qiagen, Inc., Valencia, Calif.), quantified, and fragmented at 94° C. for 35 minutes in the presence of 1× fragmentation buffer. Fragmented cRNA was hybridized to Affymetrix U133A gene chip, overnight at 42° C. Hybridization cocktail was prepared as described in the Affymetrix technical manual. Total RNA was the starting point for all replicate experiments.

dChip V1.3 software was used to generate probe level signal intensities and for normalization of data across arrays, including the replicate experiments. This program normalizes the gene expression data to one standard array that represents a chip with median overall intensity from a predetermined set of experiments. Quality metrics, including median intensity, % probe set outliers, and % single probe outliers for each chip, were used in conjunction with alignment checks to identify poor-quality chips. The chips were normalized to a baseline chip chosen by dChip with median signal. After normalization, model-based expression index signal intensity values for the chips were fit by the perfect match only model in dChip.

Statistical Analysis: Two-tailed Student's t tests were run using Microsoft Excel 2007 in order to determine statistical significance between the growth rates of parent and HNO cells treated under identical conditions. P<0.05 was considered statistically significant.

### Example 2

### Characterization of Long Term High NO (HNO) Adapted Cultures

It has been discovered that patients presenting with (or converting to), tumors with high levels of Nitric Oxide tended to do more poorly than those patients which had low expressing tumors. From that observation, cell lines were produced that were comparatively high Nitric Oxide (HNO) expressers. These HNO cell lines simulate the long term exposure of tumor cells to Nitric Oxide over the course time starting from the initial focus of cells to the late stages of disease. The High Nitric Oxide (HNO) cell lines thrive in concentration of Nitric Oxide donor that are lethal to parental cell lines. The HNO cells lines are also capable of long term maintenance in culture.

The cells lines, described below, were adapted to the nitric oxide donor DETA-NONOate. This donor was chosen based on its long half-life (approximately 24 h at 37° C. and pH 7.4), free radical mode of delivery, and rate of delivery (donation of 2 mol of NO per 1 mol of donor).

Growth Assays: The cell lines were exposed to varying doses (0-600 μM) of DETANONOate for 72 h to determine the minimum concentration of DETA-NONOate that was lethal to the cells. Each of the cell lines exhibited similar results: cell death increased with increasing concentrations of NO, and 600 μM was the minimum concentration studied that was found to be completely lethal to the cells. Therefore, 600 μM was chosen as the adaptation end-point for each of the cell lines.

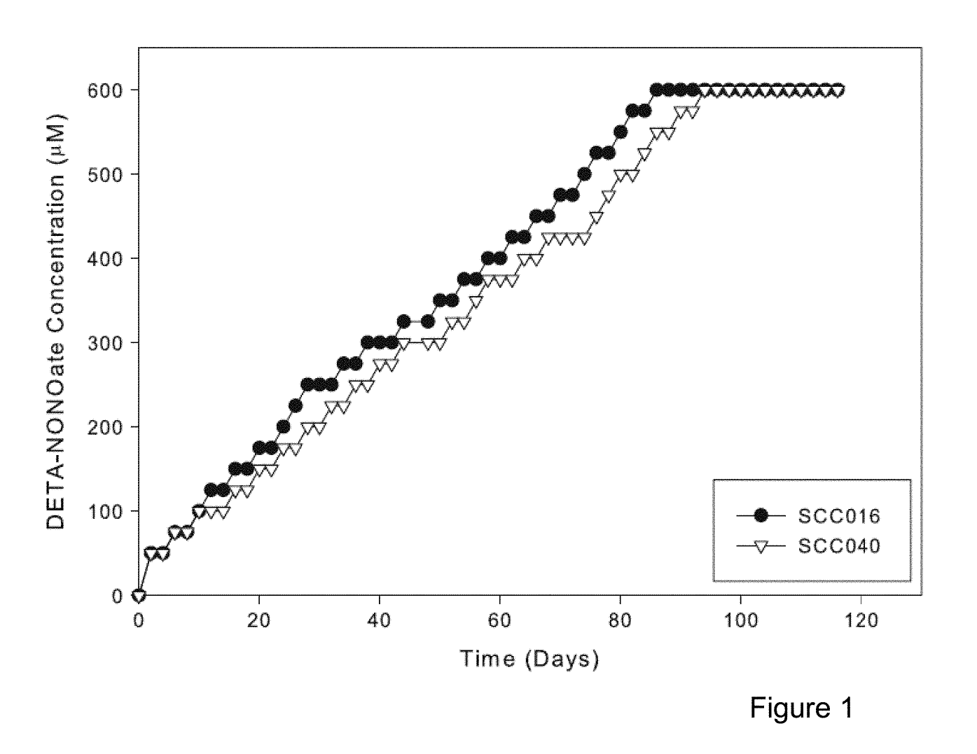

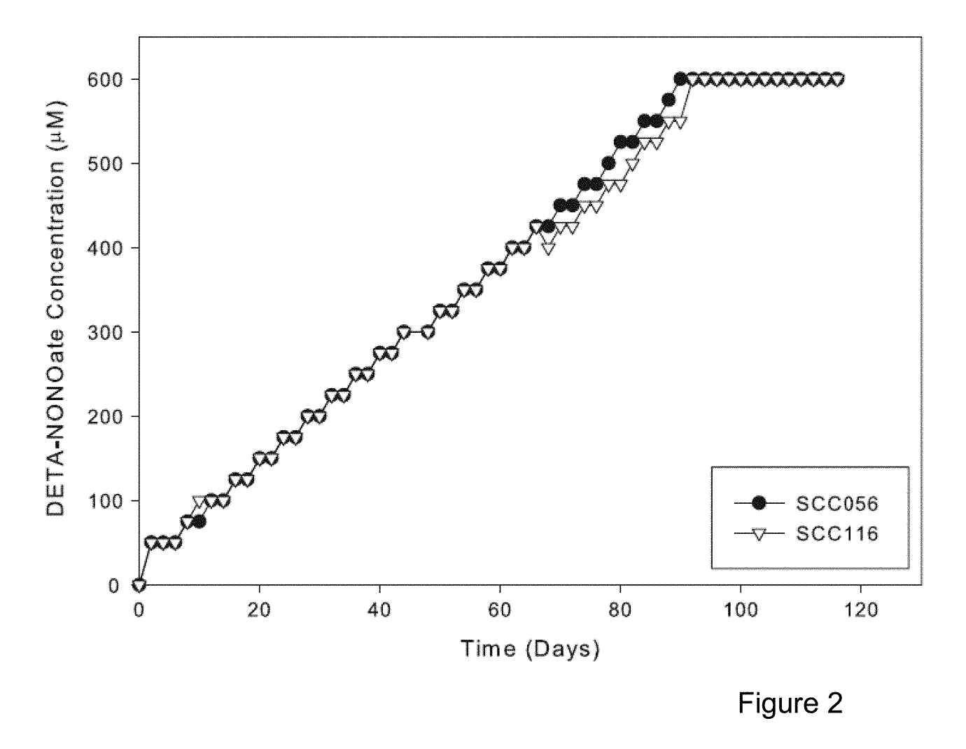

Exposure to new levels of NO donor (i.e., each 25 μM increase) typically resulted in an initially slower growth rate; however, the cell lines studied herein recovered and eventually grew robustly at all concentrations studied. FIGS. 1 and 2 show the adaptation curves for four of the human squamous head & neck cell lines (HNSCC) cell lines adapted herein. Each data point on the growth curve represents the addition of fresh DETA-NONOate solution to the growth media, either as a passaging event or as replenishment of the donor. Little difference was observed in the adaptation times for the five HNSCC cell lines: SCC016 reached 600 μM DETA-NONOate in approximately 85 days, SCC056 and SCC116 in 90 days, SCC040 and SCC114 in 95 days. SCC114 had an identical adaptation curve to SCC040, and was not shown in the curves.

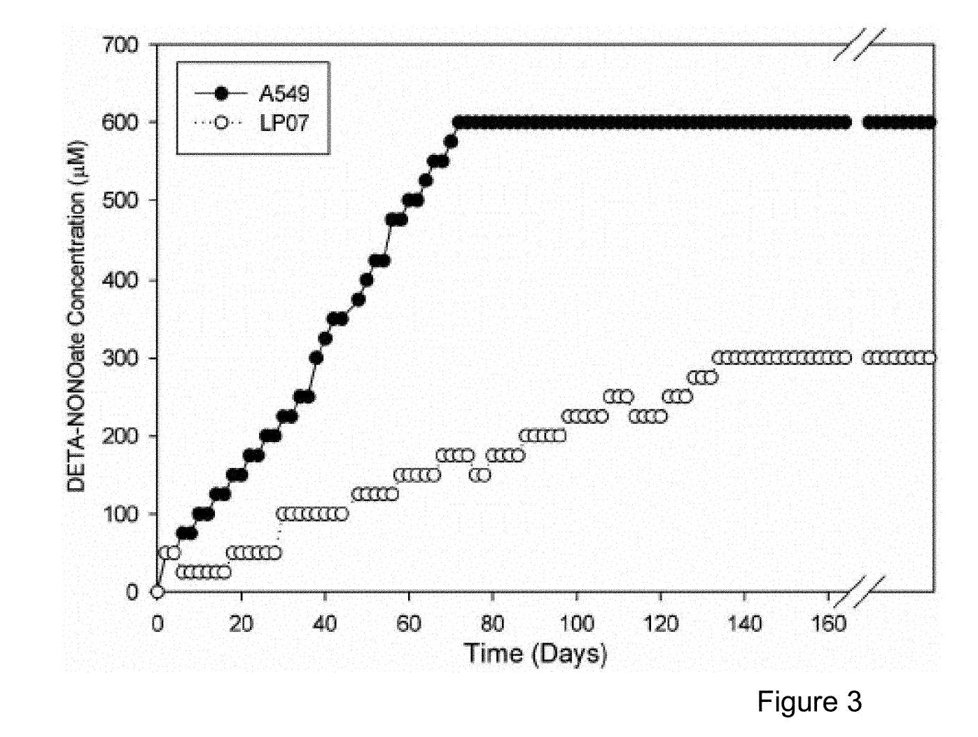

Similarly, the A549 lung adenocarcinoma cell line adapted quickly, reaching 600 μM in approximately 65 days (FIG. 3). The cells were able to thrive with each subsequent DETA-NONOate addition; at no time during the adaptation did the concentration have to be reduced. In stark contrast, the mouse tumor cell line, LP07, adapted very slowly. The adaptation process was stopped upon reaching 300 μM DETA-NONOate (FIG. 3), as the adaptation time for the LP07 cell line was greater than double that of the A549 cells. It took approximately 135 days for the cells to reach 300 μM DETA-NONOate. High levels of cellular toxicity were observed with this cell line when increased NO donor concentrations were attempted, making it frequently necessary to reduce the DETA-NONOate concentration during the adaptation process.

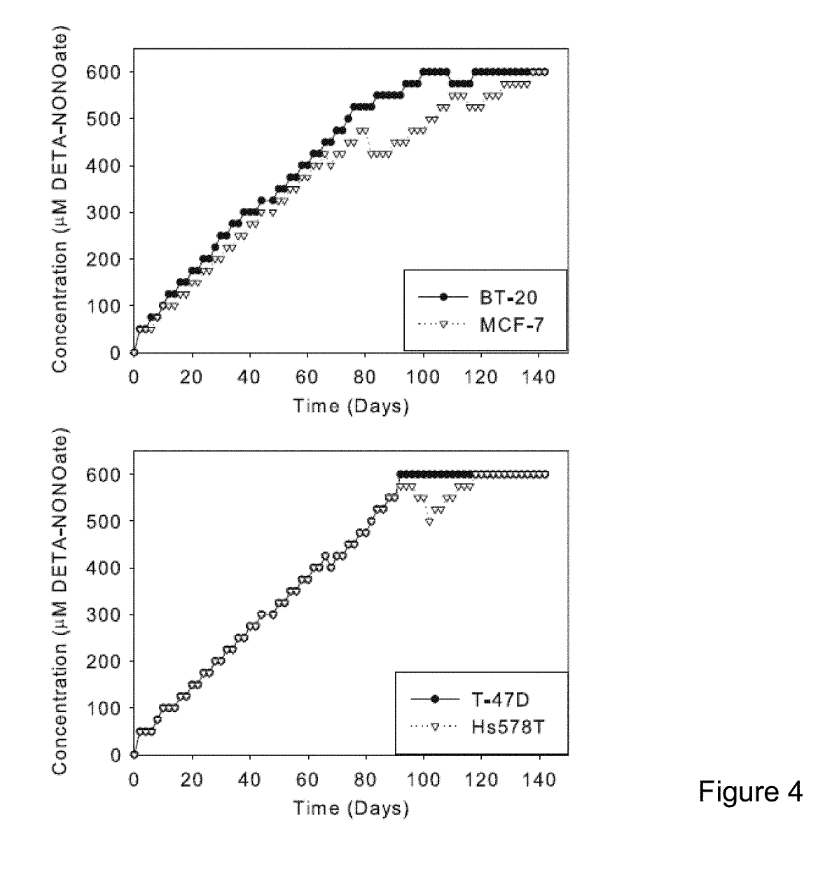

Four breast tumor cell lines (Hs578T, BT-20, T-47D, and MCF-7) were also adapted in media supplemented with 50 μM DETA-NONOate (the NO donor was added to the media immediately prior to the cells being transferred into the flask), see FIG. 4. The presence of the donor initially slowed cell growth, but the cells eventually recovered and were able to grow robustly. Among the four cell lines adapted, T-47D (˜90 days) and BT-20 (˜100 days) reached 600 μM the fastest, while Hs578T (120 days) and MCF-7 (˜140 days) each took longer to adapt. In general, cell growth was sometimes slowed upon increased donor concentration; however, at each concentration where this occurred, the cells eventually recovered and were able to grow robustly. More pronounced toxicity was observed in rare cases for both Hs578T and MCF-7. In these instances, it was necessary to temporarily decrease the amount of NO donor applied, allowing the cells an opportunity to re-acclimate to the lower concentration before attempting to raise the concentration again.

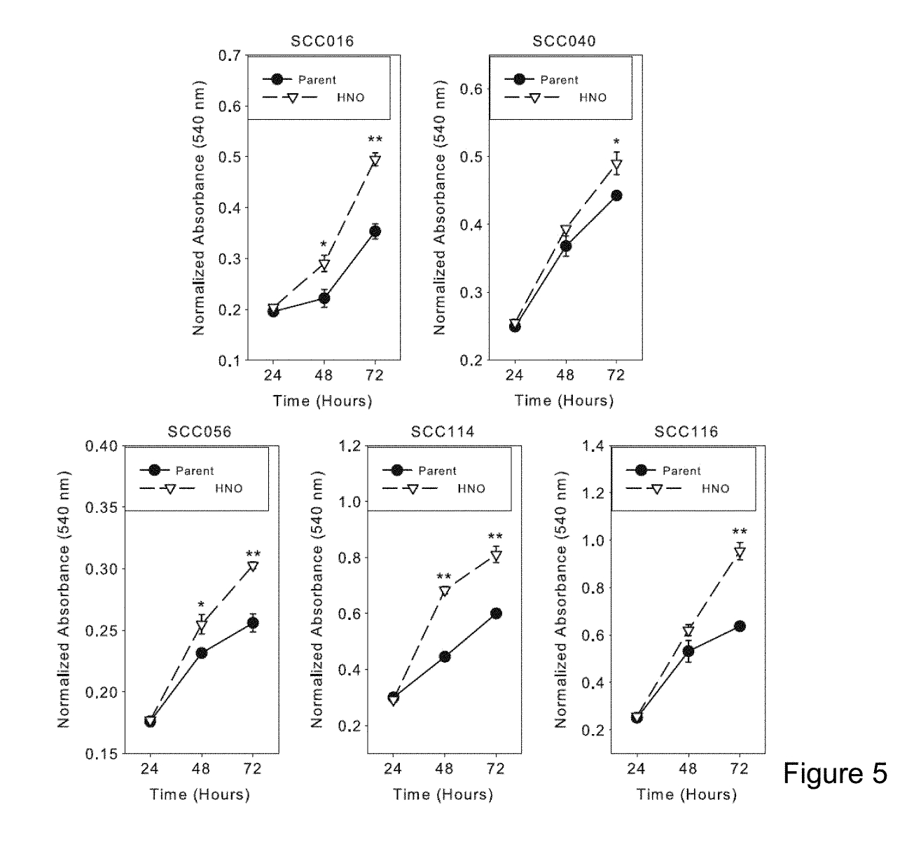

In all five HNSCC cell lines, the parent cells exhibited consistent growth in media lacking the DETA-NONOate as seen in FIG. 5, but were completely killed in media containing 600 μM DETA-NONOate. In contrast, the HNO cells were able to grow robustly in media with or without the NO donor added. FIG. 5 shows the results of cell viability/proliferation of the parent cells in standard media and HNO cells in standard media supplemented with 600 μM DETA-NONOate. Each of the HNO cell lines was found to grow faster than the corresponding parent cell line.

To verify that the enhanced growth of the HNO cell lines were inherent in the cell lines created and not attributed merely to the DETA-NONOate serving as a growth stimulant, the HNO cell lines were grown in media in which the NO donor had been removed. No significant difference was observed between the growth curves of HNO cells grown in media with DETA-NONOate versus HNO cells grown in media without donor after 72 h, confirming the HNO cells grow more aggressively than their corresponding parent cells. Data now shown.

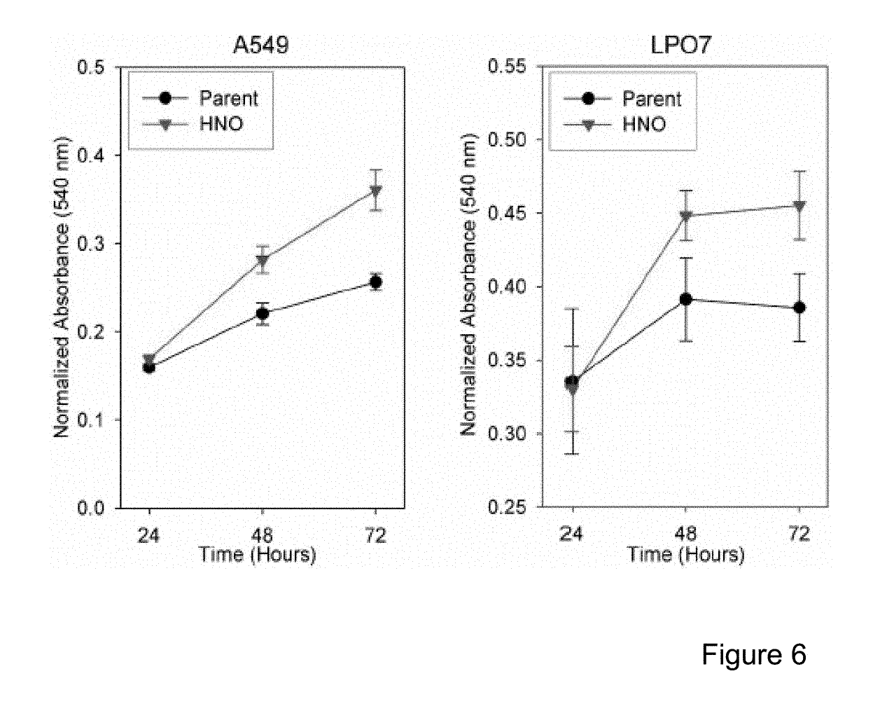

To verify the chosen end-point of the breast adenocarcinoma, the A549-HNO cells were grown in media in which the NO donor was removed; the parent A549 cells were grown in media containing 600 μM DETA-NONOate. As expected, the parent cells were not able to survive when placed directly into a HNO environment of 600 μM donor (FIG. 6). Removal of the NO donor source from A549-HNO cells did not affect growth over the 72 hour period measured. Analogous results were obtained for the LP07 and LP07-HNO cell lines at a DETA-NONOate concentration of 300 μM.

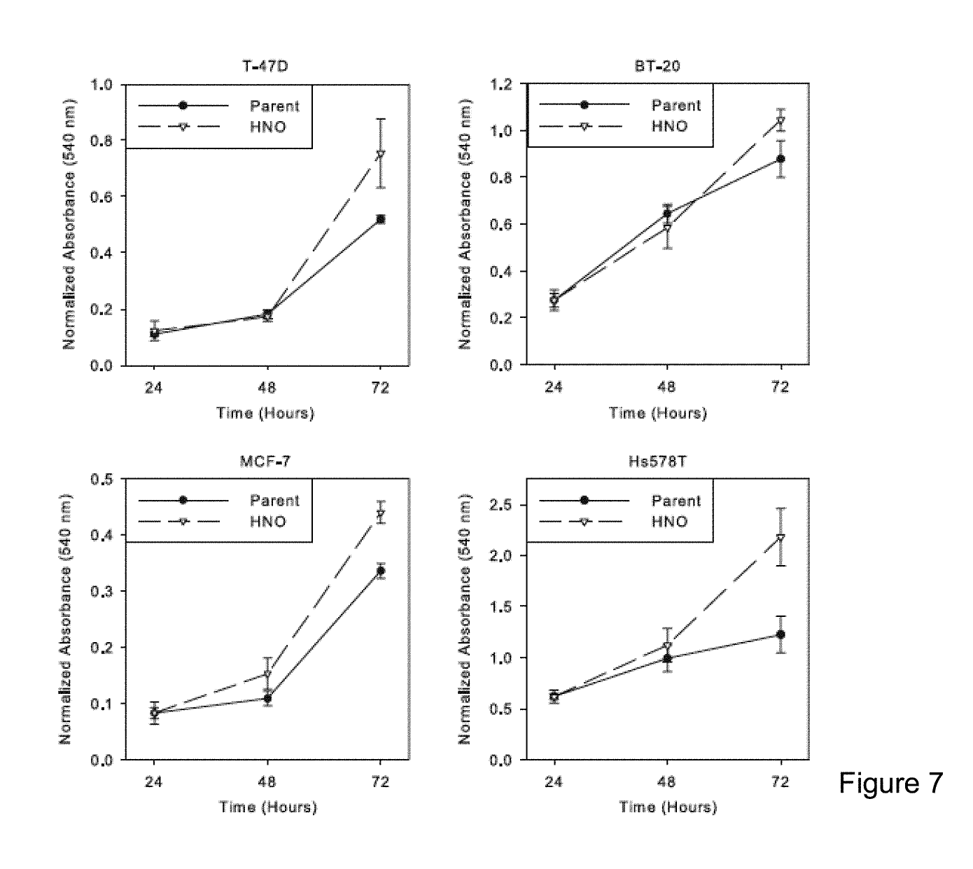

Cell viability/proliferation assays of the parent cells (in standard media) and the HNO cells (in standard media supplemented with 600 μM DETA-NONOate) are shown in FIG. 7. For each parent/HNO pair, the HNO cell line was found to grow faster than the corresponding parent cell line at 72 h. Hs578T showed the largest difference in cell growth between the parent and HNO cell lines, with the HNO cells growing nearly twice as fast as the parent cells after 72 h. BT-20 showed the smallest difference, with a nearly 20% increase in HNO cell growth versus the parent at 72 h.

Similar to experiments carried out in the adaptation of the A549 human lung cell line in FIG. 6, the breast cancer HNO cells were grown in media in which the NO donor had been removed. The purpose of this experiment was to verify that the enhanced growth of the HNO cell line was inherent in the cell line created, and not attributed merely to the DETANONOate serving as a growth stimulant. After 72 h, little difference was observed between the growth curves of HNO cells growing in media with DETA-NONOate versus HNO cells grown in media without donor, confirming that the HNO breast cancer cell lines also grow more aggressively than their corresponding parent cells.

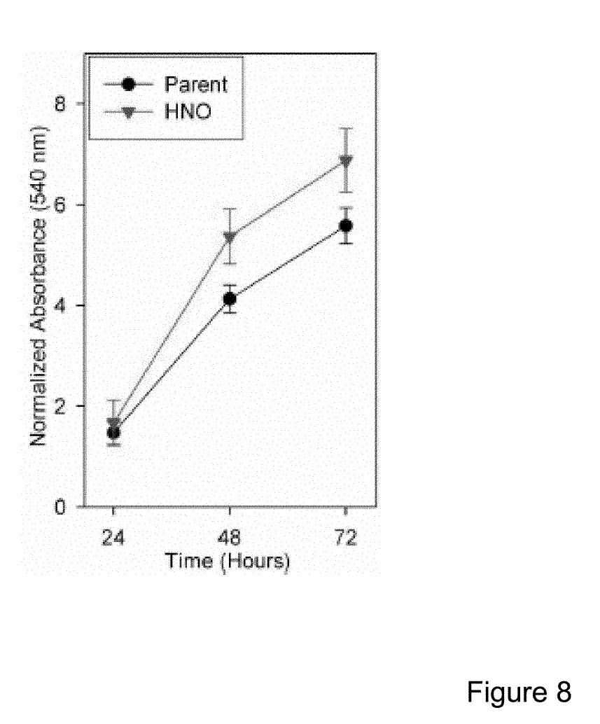

Growth curves of the parent and adapted cells were also compared in serum-less media (FIG. 8). The A549-HNO cells and the LP07-HNO cells grew at measurably faster rates than their corresponding parent cells.

The FACS sorted number of cells that were positive to the uptake of Hoechst 33342 dye is shown in Table 1. It should be noted that this is the minimum number as over time, both the tumor cells, and tumor stem cells will become positive.

TABLE 1
Fluorescence-activated cell sorting (FACS) for Hoechst 33342 dye

| Cell Line | Number of Cells Positive for Hoechst 33342 |
|---|---|
| A549 Parent | 14,944 +/− 15,000 |
| A549 HNO | 5663 +/− 15,000 |

TABLE 2
Cell surface markers found on the HNO cell lines.

| Cell Line | CD24 | CD34 | CD38 | CD44 | CD133 | CD166 |
|---|---|---|---|---|---|---|
| A549 P | 0 | 0 | ++ | 0 | ++ | +++ |
| A549 HNO | 0 | 0 | ++ | + | + | +++ |
| T47D P | 0 | 0 | + | + | 0 | +++ |
| T47D HNO | ++ | 0 | +++ | ++ | ++ | +++ |
| Hs578T P | 0 | 0 | + | ++ | 0 | ++ |
| Hs578T HNO | 0 | 0 | +++ | +++ | 0 | ++ |
| MCF7 P | +++ | 0 | ++++ | ++ | + | ++++ |
| MCF7 HNO | ++ | 0 | ++++ | +++ | +++ | ++++ |
| BT20 P | + | 0 | ++++ | +++ | 0 | ++++ |
| BT20 HNO | 0 | 0 | +++ | +++ | ++ | +++ |
| SCC016 P | 0 | +++ | +++ | + | ++ | ++++ |
| SCC016 HNO | 0 | 0 | ++++ | ++ | +++ | ++++ |
| SCC040 P | 0 | 0 | ++ | +++ | +++ | ++ |
| SCC040 HNO | 0 | 0 | ++++ | +++ | +++ | ++++ |
| SCC056 P | 0 | 0 | +++ | +++ | +++ | +++ |
| SCC056 HNO | 0 | 0 | 0 | ++ | 0 | ++++ |
| SCC114 P | 0 | 0 | ++ | ++ | 0 | +++ |
| SCC114 HNO | 0 | 0 | ++++ | ++++ | + | ++++ |
| SCC116 P | 0 | 0 | ++ | + | 0 | +++ |
| SCC116 HNO | 0 | 0 | +++ | ++ | ++ | +++ |

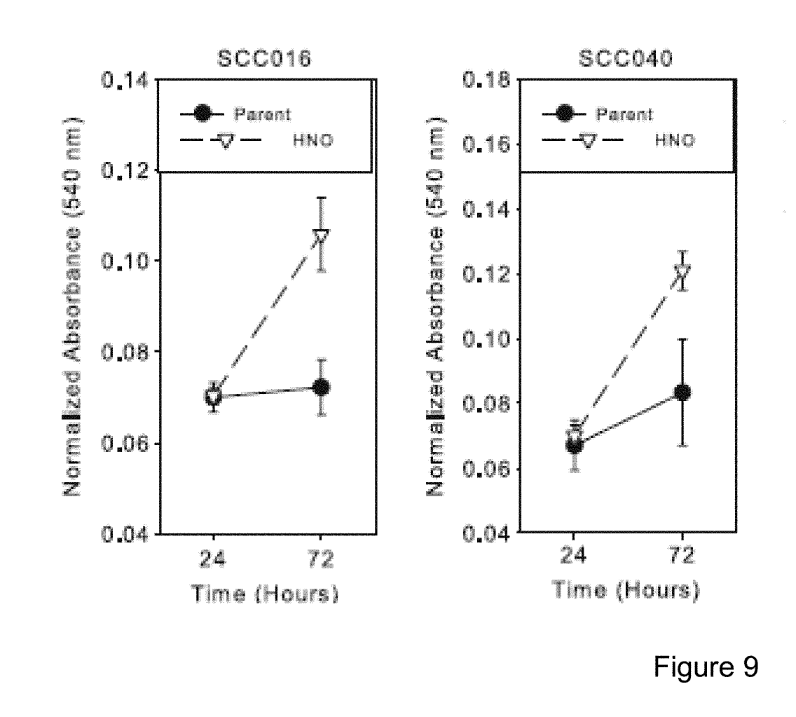

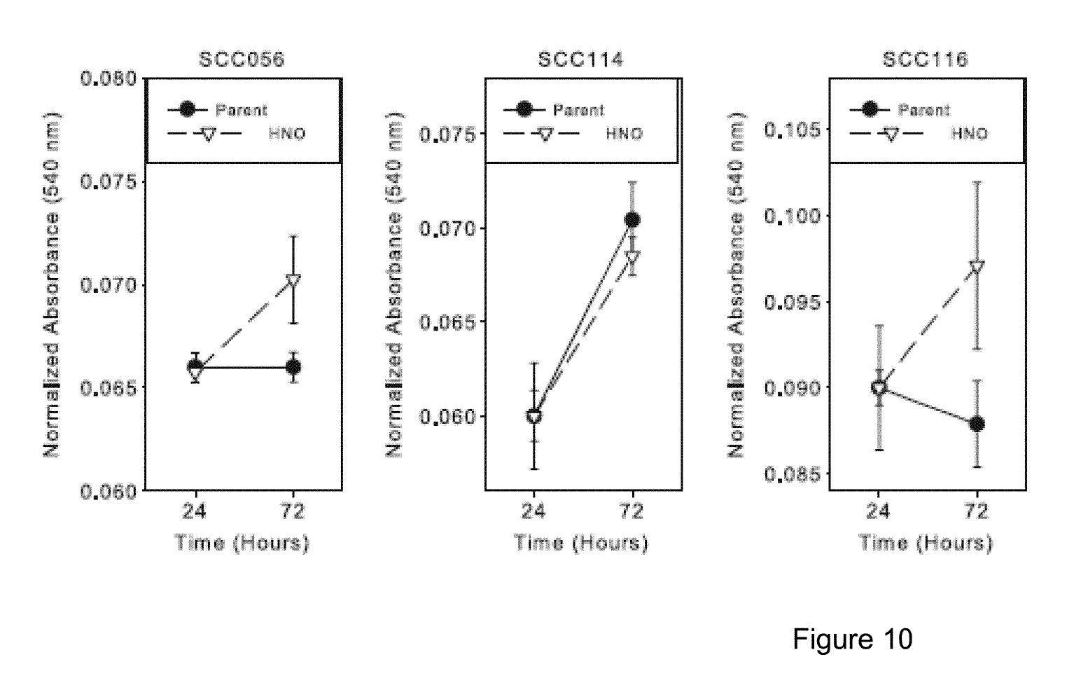

Growth On Soft Agar: The ability of tumors to grow in low nutrient growth media such as growth on soft agar is used to measure a tumor's aggressiveness. FIGS. 9 and 10 show growth curves for the five HNSCC cell lines (parent and HNO adapted cell lines) on soft agar. Four of the five HNO cell lines were found to grow faster than their corresponding parent cell lines on soft agar; only SCC114 exhibited similar growth rates between parent and HNO cells.

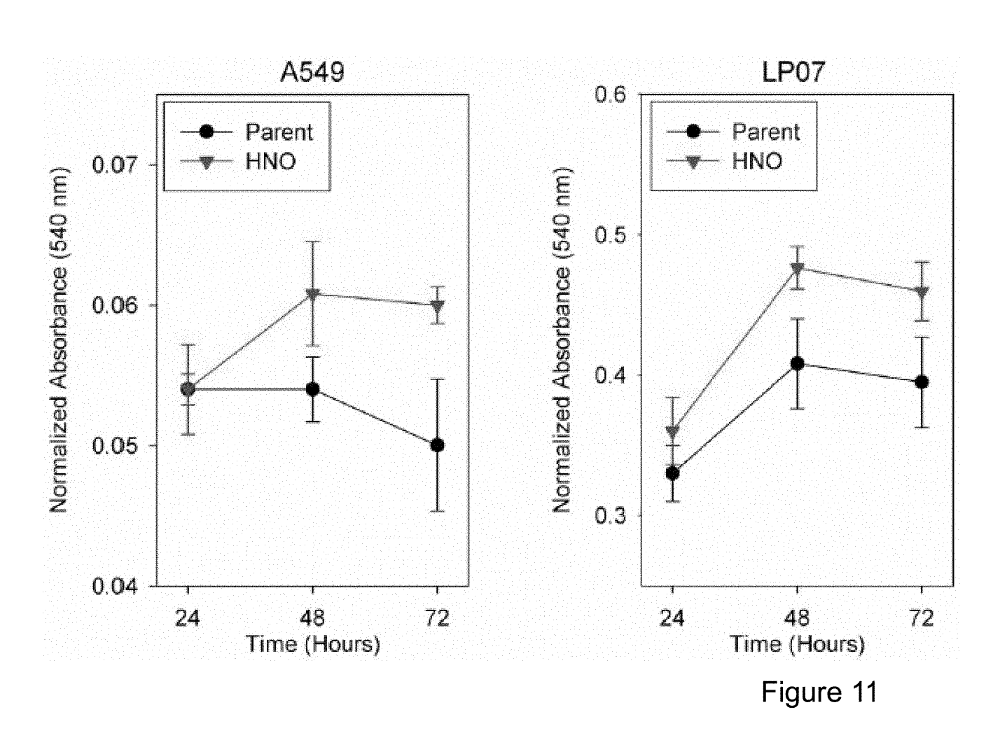

The A549-HNO cells also grew at a measurably faster rate than their corresponding parent cells. Similar results were observed for the LP07-HNO cells grown on soft agar. (See FIG. 11.)

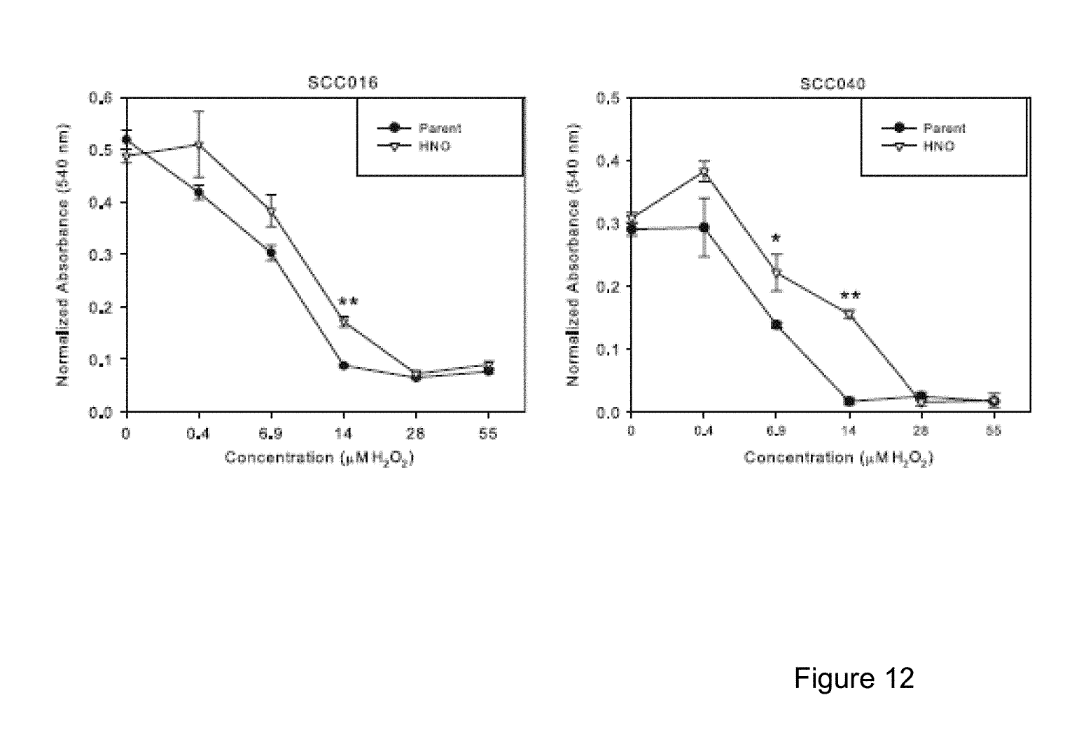

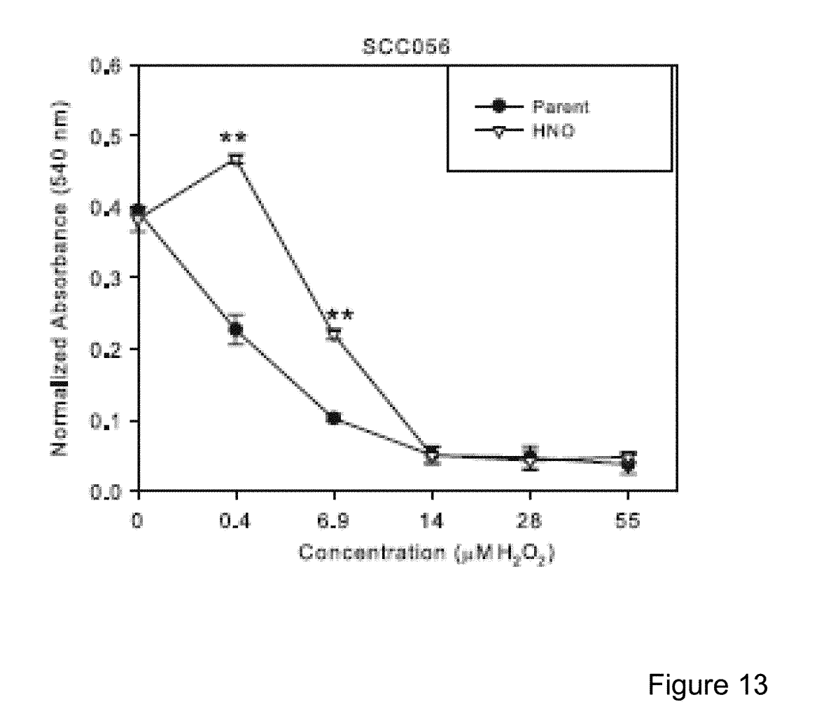

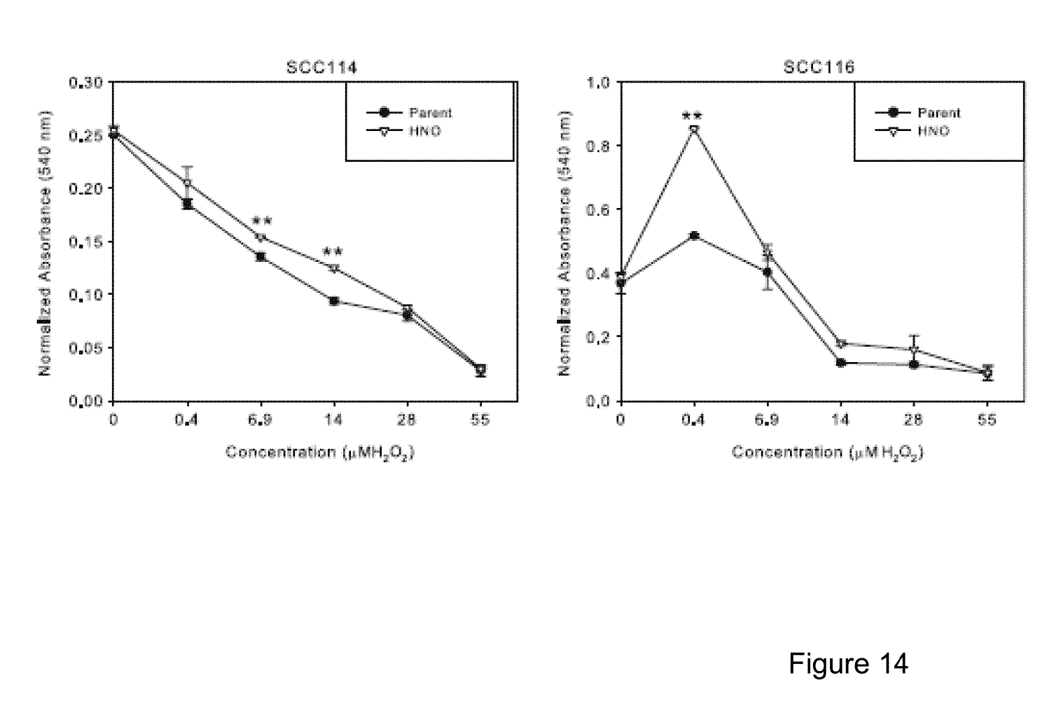

Exposure to H₂O₂: Parent and HNO cells were grown in the presence of hydrogen peroxide to test if the cells could survive a high free radical environment generated by an oxygen-based donor. Comparatively high concentrations of H₂O₂ (above 55 μM) killed both parent and HNO cells upon 24 h exposure [data not shown]; however, at lower concentrations (0.4-14 μM), each of the five HNO cell lines exhibited a statistically significant (P<0.05) higher cell viability than their corresponding parent cell line at one or more of the concentrations studied (see FIGS. 12-14).

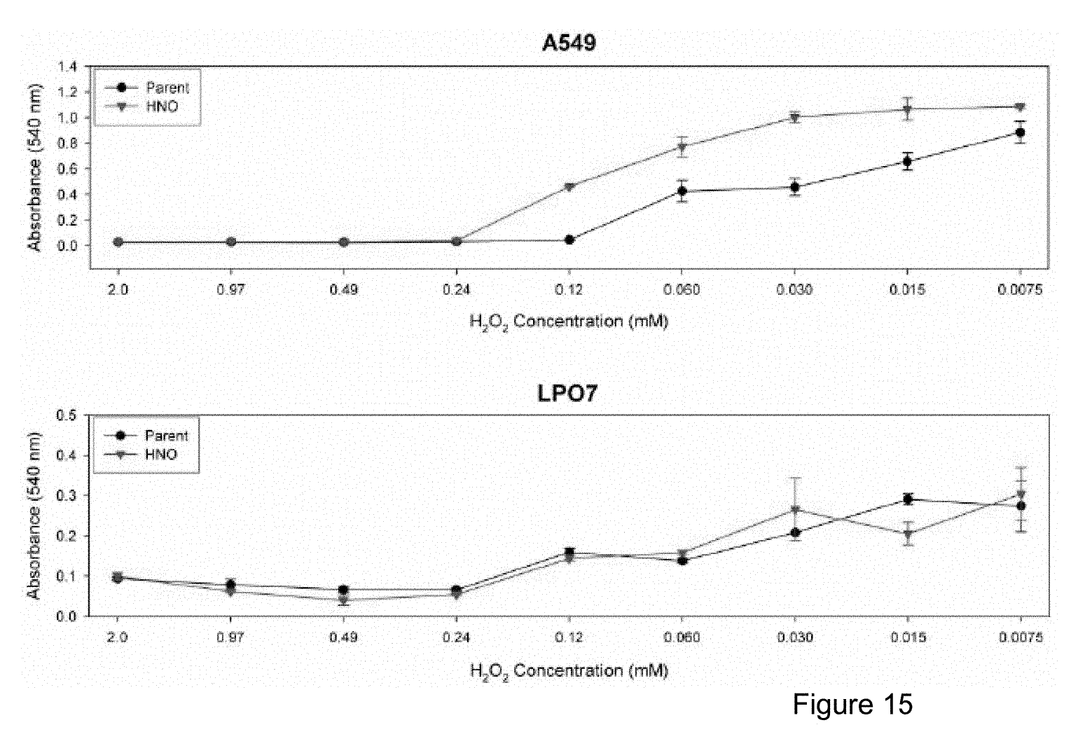

In FIG. 15, the A549 cell lines, both A549 parent and A549-HNO cells, were killed at concentrations of 0.055 mM H₂O₂ and above. However, at concentrations of 0.028 mM and below, the A549-HNO cells exhibited far greater viability/proliferation than their corresponding parent cells. In contrast, the LP07 and LP07-HNO cells exhibited similar viability/proliferation at all concentrations of H202 tested. See FIG. 15.

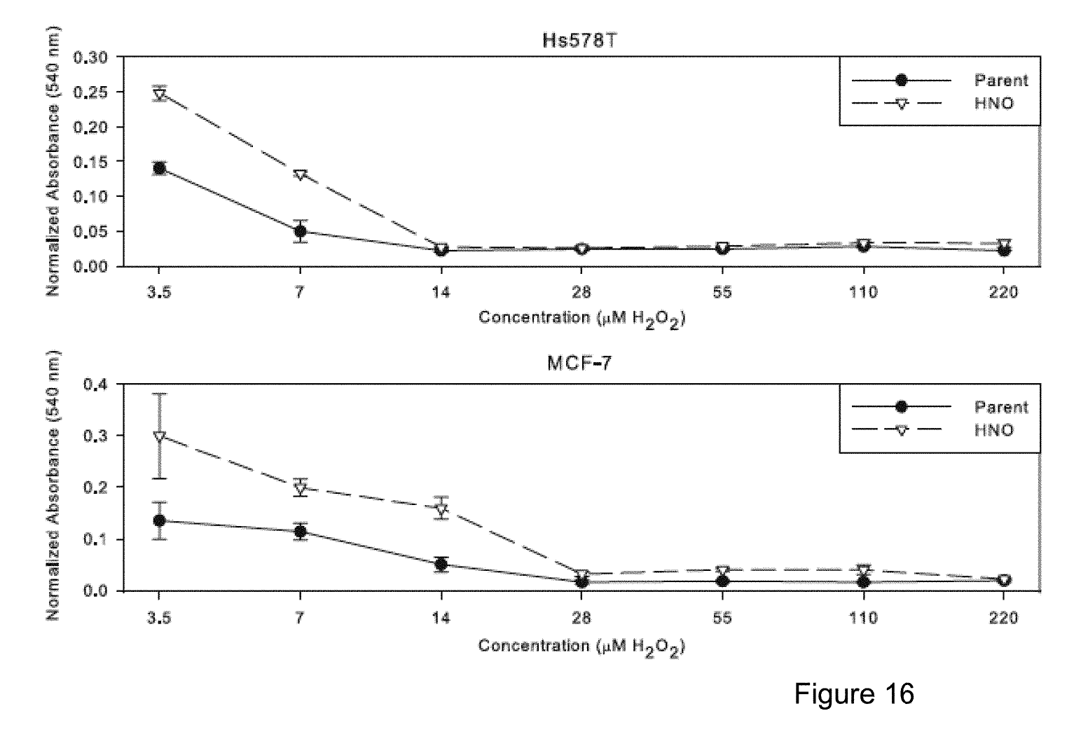

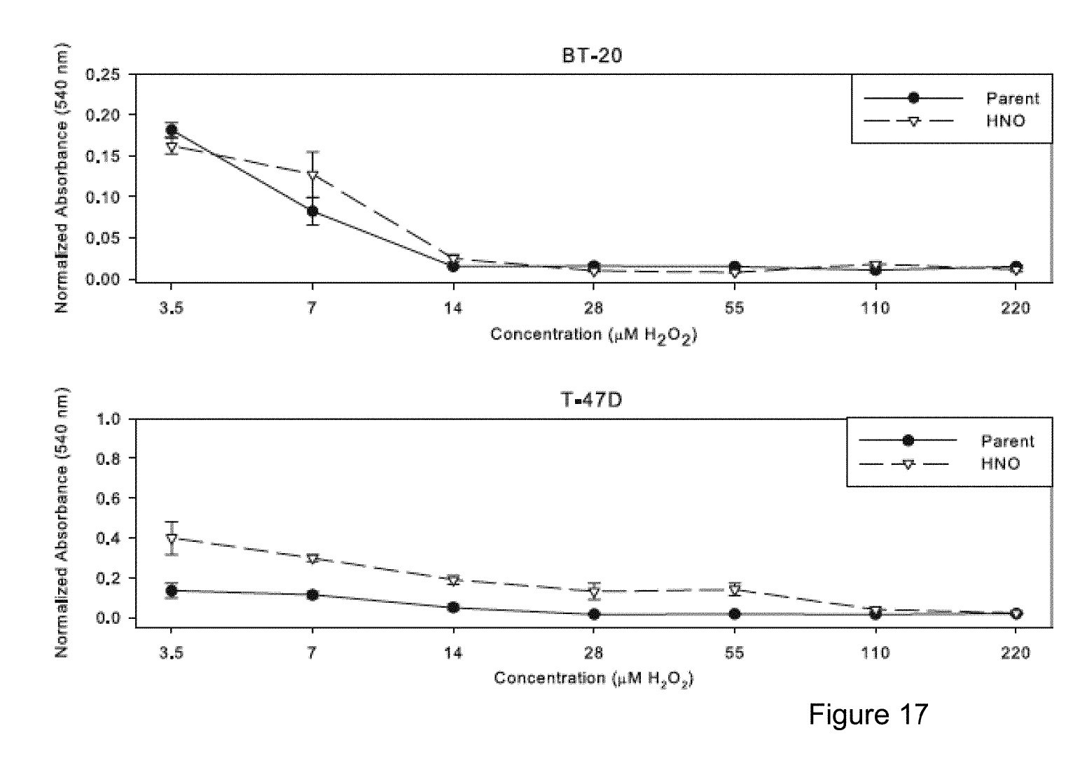

All four breast adenocarcinomas cell line pairs, both the parent and HNO cells were killed at high concentrations (110 μM and above) of H₂O₂ shown in FIGS. 16 and 17. However, at lower concentrations (7 μM and below), the HNO cell line showed increased growth over the corresponding parent cell line for three of the four cell line pairs (T-47D, Hs578T, and MCF-7). Parent and BT-20-HNO cells showed similar growth behavior at all concentrations studied.

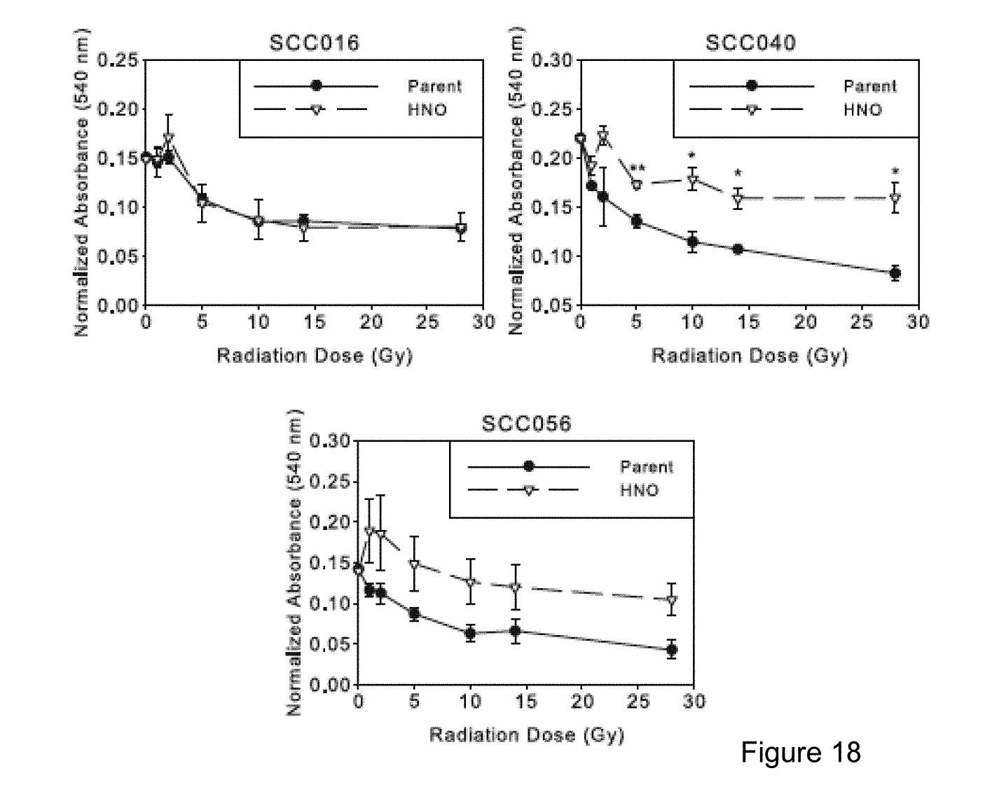

Radiation Resistance: Growth assays were used to compare the ability of the parent and HNO cells to withstand X-ray and UV radiation. The results of the X-ray radiation studies are shown in FIG. 18. Like the UV results (discussed below), no difference in viability was observed between SCC016 parent and SCC016-HNO at all X-ray doses studied. Unlike the UV results, however, SCC040-HNO exhibited significantly higher viability than the SCC040 parent cells at the higher doses studied. The SCC056 cell line showed a similar trend, with the SCC056-HNO cells exhibiting greater viability than their corresponding parent cells. The SCC056-HNO cells were nearly completely resistant to doses of 5 Gy and below, and only limited toxicity (˜10-20% cell death) was observed at the higher doses tested.

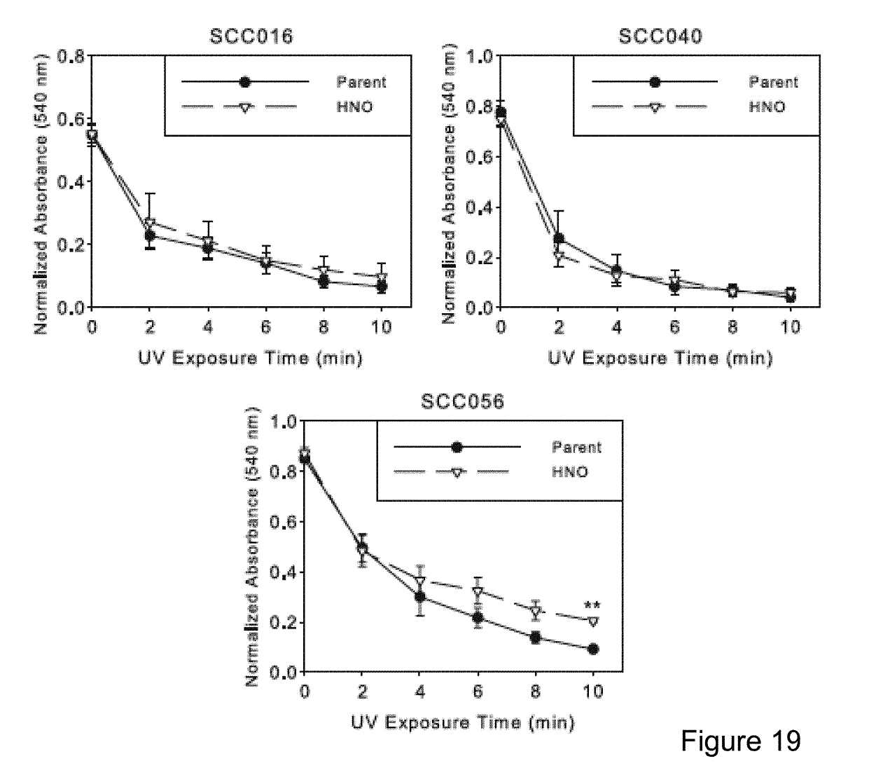

UV Resistance: FIG. 19 shows the results of the UV radiation studies. In both the SCC016 and SCC040 cell line pairs, similar responses to the UV treatment were observed: extreme toxicity was observed upon exposure to UV light at all time periods tested. The observed toxicity was especially high at longer exposure times (8 and 10 min). For these two cell line pairs, no difference was observed between the parent and HNO cells upon UV treatment. In contrast, the SCC056 cell line pair showed a better general tolerance of the UV light than the other two cell line pairs studied (i.e., a lower percentage of cell death), and a difference was observed between the parent and HNO cell lines. The SCC056-HNO cell line exhibited greater cell viability than the SCC056 parent cell line at the longer exposure times.

Cell Migration: Cell migration activity was measured via a CytoSelect Assay™ over a 2-24 hour period using a commercial assay system sold by Cell Biolabs, Inc. HNO adapted cells migrated faster and more robustly than the parental cell lines.

FACS Cell Cycle Analysis: Using PI staining and FACS cell cycle analysis, HNO cell lines were confirmed as having a higher S phase population than the parent cell lines. This was consistent with the cells growing faster. In addition, no aneuploidy, or change in aneuplody was noted using FACS analysis between the parent and the HNO cell lines.

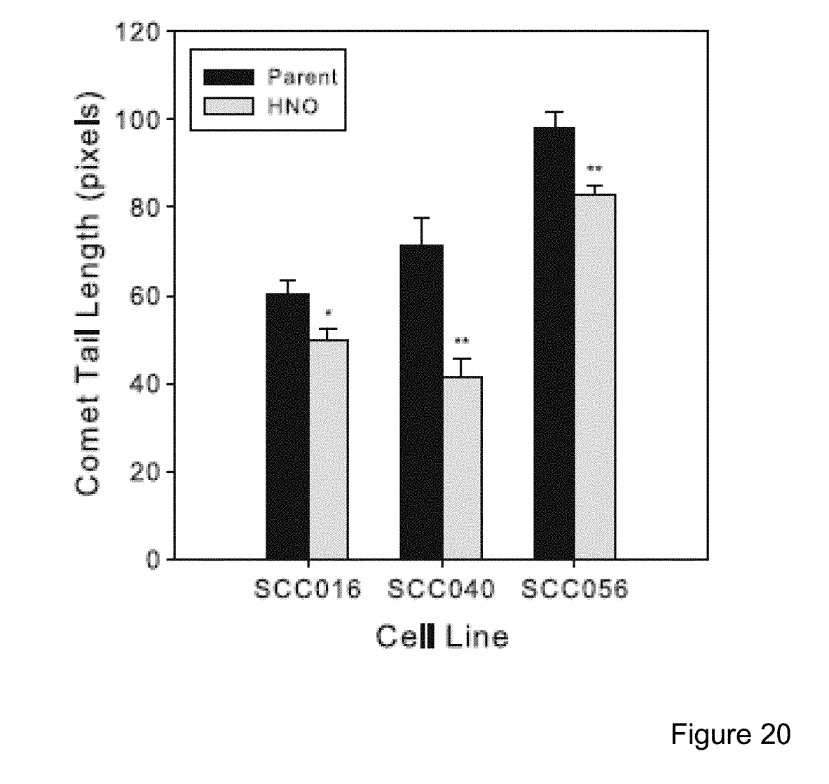

COMET Assays: Single cell gel electrophoresis (CometAssay™) was carried out to quantify the amount of DNA damage in the three parent/HNO cell line pairs (FIG. 20). In each cell line, the parent cells exhibited longer Comet tails than their corresponding HNO cells, with the SCC040 cell line pair exhibiting the greatest difference in Comet tail length between parent and HNO cells. The SCC056 cell line pair (both parent and HNO cells) exhibited longer Comet tail lengths than were observed in the other two cell line pairs. Overall, the comet tails were consistently shorter for the HNO cell lines, indicating that they performed consistent, active DNA repairs. These data were consistent with the radiation and UV exposure DNA.

Immunoblotting and Immunohistochemistry: Thirty-four specimens of HNSCC cell lines were stained using antibodies for eNOS, iNOS, GST-pi, and APE1. Sections processed without the primary antibody served as negative controls and showed no staining. Table 3 shows the breakdown of the staining intensity for iNOS, eNOS. Nearly all samples (33 of 34, 97.1%) exhibited at least some degree of iNOS expression, with 27 of the 34 (79.4%) exhibiting moderate-to-strong staining. In contrast, no immunostaining for eNOS was observed in 15 samples (44.1%) and only weak staining was seen in 14 of the 19 positive samples (73.7%). Nineteen cases expressed both isoforms of NOS, while only one case was negative for both. The staining for eNOS and iNOS was cytoplasmic in all positive cases. Cytoplasmic GST-pi expression was observed in all 34 cases, with the majority 76.5%) exhibiting strong staining. Cytoplasmic APE1 expression was found in nearly all specimens (33 of 34, 97.1%) and the majority of samples exhibited moderate or strong staining (24 of 34, 70.6%). The level of iNOS, GST-pi, and/or APE1 expression did not show significant correlation with patient age/sex, smoking/drinking history, tumor size, tumor grade, or TNM stage in this limited sample size.

TABLE 3
Number of clinical specimens observed at each staining intensity
(0-3+) for iNOS, eNOS, GST-pi, and APE1 (n = 34).

| Staining Intensity | iNOS | eNOS | GST-π | APE1 |
|---|---|---|---|---|
| 3+ | 14 | 1 | 26 | 9 |
| 2+ | 13 | 4 | 5 | 15 |
| 1+ | 6 | 14 | 3 | 9 |
| 0 | 1 | 15 | 0 | 1 |
| Total Cases | 34 | 34 | 34 | 34 |

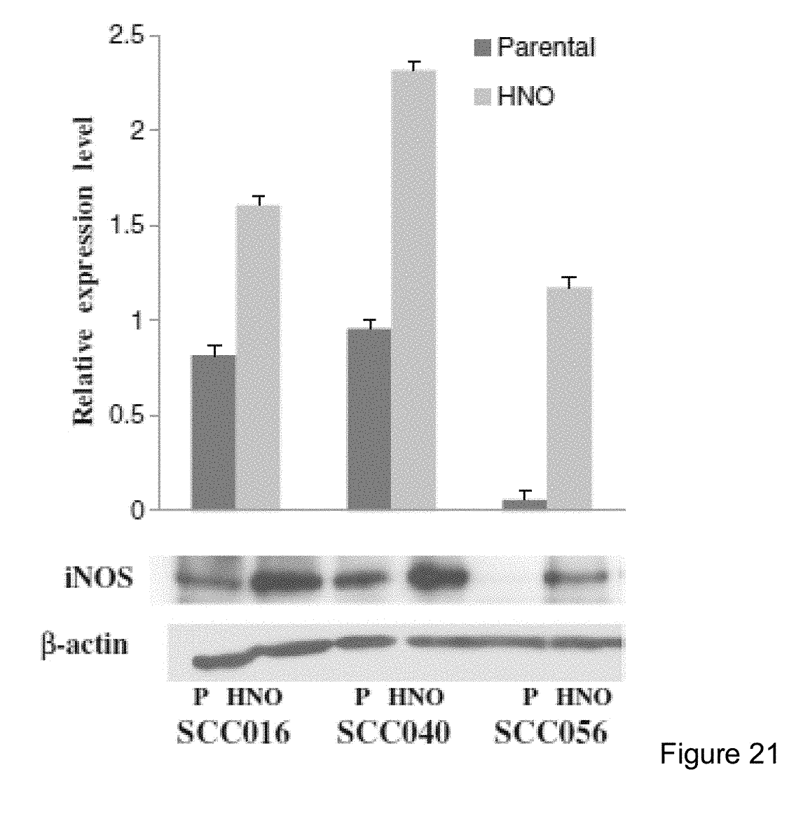

Western blot analysis was used to detect the expression levels of two NOS isotypes: iNOS and eNOS. As shown in FIG. 21, for each cell line pair, iNOS expression was much greater in the HNO tongue squamous carcinoma cell line than in the corresponding parent cell line. SCC040-HNO exhibited the greatest iNOS expression, whereas the SCC056 parent cell line showed only minimal expression. In contrast to the iNOS expression, no eNOS expression was detected in any of the three tongue carcinoma cell lines—in either parent or HNO cells

Table 4 presents a summary table of the cell cultures, and indicates that the HNO cell lines are much more aggressive in their biological properties. This is consistent with an enriched population of tumor stem cells, and is reflective of the Nitric Oxide clinical findings. In addition, the HNO cell lines (and not the parent cell lines) that have been tested to date, express two properties that have been used to define tumor stem cells: 1) positive expression of Aldehyde Dehydogenase-1 (ALDH-1), and 2) enriched populations of Hoechst 33342 negative populations (as high as 50%).

TABLE 4
Biological properties of the HNO-adapted cell lines, relative to their
parental cell line.

| | Lung | Head & Neck | | | | | Breast | | | |
|---|---|---|---|---|---|---|---|---|---|---|
| | A549 | SCC016 | SCC040 | SCC056 | SCC114 | SCC116 | T-47D | Hs578t | BT-20 | MCF-7 |
| Rapid | +++ | +++ | +++ | +++ | +++ | +++ | +++ | +++ | +++ | +++ |
| Growth | | | | | | | | | | |
| Serum-less | +++ | +++ | +++ | +++ | +++ | +++ | N/D | N/D | N/D | N/D |
| Growth | | | | | | | | | | |
| Soft Agar | + | + | + | + | + | + | ++ | ++ | ++ | +++ |
| Growth | | | | | | | | | | |
| ecNOS | + | − | +++ | ++ | + | − | N/D | N/D | N/D | N/D |
| expression | | | | | | | | | | |
| GST-pi | ++ | ++ | + | + | + | ++ | N/D | N/D | N/D | N/D |
| expression | | | | | | | | | | |
| APE-1 | ++ | ++ | − | ++ | + | + | N/D | N/D | N/D | N/D |
| expression | | | | | | | | | | |
| ALDH | + | N/D | N/D | N/D | N/D | N/D | + | + | + | + |
| expression | | | | | | | | | | |
| COMET | +++ | ++ | +++ | ++ | + | − | N/D | N/D | N/D | N/D |
| Tail Length* | | | | | | | | | | |
| Radiation | +++ | − | ++ | ++ | − | +++ | + | + | − | − |
| Resistance | | | | | | | | | | |
| UV | + | − | − | + | + | + | + | + | − | + |
| Resistance | | | | | | | | | | |
| H2O2 | 0.055 mM | 0.028 mM | 0.028 mM | 0.028 mM | 0.55 mM | 0.028 mM | 0.028 mM | 0.0069 mM | 0.0069 mM | 0.014 mM |
| Resistance** | | | | | | | | | | |
| Temp. | 40° C. | 40° C. | 40° C. | 42° C. | 37° C. | 37° C. | 40° C. | 40° C. | 37° C. | 44° C. |
| Resistance*** | | | | | | | | | | |
| S-Phase | 25.7 | 7.7 | 6.0 | 5.2 | 6.6 | 31.7 | 5.2 | 21.6 | 14.7 | 18.3 |
| %**** | | | | | | | | | | |
| Hoechst Dye | +++ | N/D | N/D | N/D | +++ | N/D | +++ | N/D | N/D | N/D |
| Exclusion | | | | | | | | | | |

*Measured as Parent length > HNO length (i.e., +++ score: Parent tail lengths are much longer than HNO tail lengths).
**Highest concentration of H2O2 that the HNO cell lines grow in which the parent cell lines do not survive.
***Temperature in C. degrees that is the start of the difference between Parent and HNO cell lines.
****Measured as % HNO cells in S-phase minus the % Parent cells in S-phase.

GeneChip Array: Expression profiles generated from A549 parent cells compared to HNO A549a cells can be found in Appendix A.

ALDEFLUOR Assay: Aldehyde Dehydrogenase 1 (ALDH-1) activity was measured through the production of a fluorescent molecule detected in 520-540 nm range in a 96 well fluorescent plate reader or by FACS. Commercial kits were used per the manufacturer's instructions from Stemcell Technologies, Inc. ALDH-1 data is presented in Table 4.

Chemotherapy Resistance: The Hoechst 33342 staining profiles suggested a reduced number of cells were actively undergoing apoptosis, as compared to the parental cell line. Increased chemotherapy resistance would be expected since the cells have demonstrated upregulation of ABC transporter proteins, elevation in the protective mechanisms such as Glutathione S-Tranferase-pi (GST-π). and increased expression DNA repair enzymes, such as APE-1 (Apurinic/apyrimidinic endonuclease-1). Preliminary data suggest that GST-π is one of the protective mechanisms that cells use to adapt to a highly reactive environment. GST-π expression was higher in the adapted cell lines than in the parent cell lines, see Table 4. The expression levels of ecNOS were variable among the adapted cell lines. Therefore, the consistent increase in expression of GST-π was not due to generalized growth properties. These data, shown in Table 4, were consistent with COMET, UV, and radiation data.

Temperature Resistance: The HNO tumor cell lines also had a greater tolerance for increased temperature, suggesting an aggressive cell type for greater survival range. Using a PCR thermocycler, a step gradient from 35° C. to 60° C. in 5 degree (5 min/degree change) increments was used. 96 well plate volumes of cells were put into sterile PRC tubes, and placed in the thermocycler. Sets of cells were removed at each temperature, and the temperature increased. The cells were then plated in 96 well plates, and the assessed for cell viability after 24 hours using MTT. As outlined in Table 4, the HNO cell lines had higher temperature resistance than the parent cell lines.

In Vivo Growth: Animal model experiments showed that as few as 5 tumor stem cells (that were isolated by FACS sorting) gave rise to tumors in mice. The growth of xenograft tissues or tumor stem cell lines are expected to demonstrate growth of tumors from the HNO tumor stem cells. Intravenous or intraperitoneal injection of as few as 1-5 tumor stem cells (HNO cells) as opposed to several million parental cells will result in palpable tumors. The tumors will also be faster growing in the animals than the parent cell lines, and would be more invasive with earlier metastatic activity.

One skilled in the art will appreciate further features and advantages of the invention based on the above-described embodiments. By way of non-limiting example, the devices and methods of the present invention can employ raster scanning of a point source of illumination light rather than axial scanning of an elongated beam to achieve a 3-dimensional map. Accordingly, the invention is not to be limited by what has been particularly shown and described, except as indicated by the appended claims. All patents, publications and references cited herein (including the following listed references) are expressly incorporated herein by reference in their entirety.

Further understanding of various aspects of the invention can be obtained by reference to the GeneChip dataset reproduced in Appendix A and the following articles and poster presentations, which are all incorporated by reference.

Bonnet D, Dick J E (July 1997). Human acute myeloid leukemia is organized as a hierarchy that originates from a primitive hematopoietic cell. Nature medicine 3 (7): 730-7.

Matsui W, Huff C A, Wang Q, Malehorn M T, Barber J, Tanhehco Y, Smith B D, Civin C I, Jones R J (March 2004). Characterization of clonogenic multiple myeloma cells. Blood 103 (6): 2332-6.

Singh S K, Clarke I D, Terasaki M, Bonn V E, Hawkins C, Squire J, Dirks P B (September 2003). Identification of a cancer stem cell in human brain tumors. Cancer research 63 (18): 5821-8.

Al-Hajj M, Wicha M S, Benito-Hernandez A, Morrison S J, Clarke M F (April 2003). Prospective identification of tumorigenic breast cancer cells. Proceedings of the National Academy of Sciences of the United States of America 100 (7): 3983-8.

O'Brien C A, Pollett A, Gallinger S, Dick J E (January 2007). A human colon cancer cell capable of initiating tumour growth in immunodeficient mice. Nature 445 (7123): 106-10.

Zhang S, Balch C, Chan M W, Lai H C, Matei D, Schilder J M, Yan P S, Huang T H, Nephew K P (June 2008). Identification and characterization of ovarian cancer-initiating cells from primary human tumors. Cancer research 68 (11): 4311-20.

Li C, Heidt D G, Dalerba P, Burant C F, Zhang L, Adsay V, Wicha M, Clarke M F, Simeone D M (February 2007). Identification of pancreatic cancer stem cells. Cancer research 67 (3): 1030-7.

Maitland N J, Collins A T (June 2008). Prostate cancer stem cells: a new target for therapy. J. Clin. Oncol. 26 (17): 2862-70.

Lang Sh, Frame F, Collins A (January 2009). Prostate cancer stem cells. J. Pathol. 217 (2): 299-306.

England's National Institute of Health and Clinical Excellence. Full guidance on the use of paclitaxel in the treatment of ovarian cancer, 22 January 2003.

Mor, G. Yale Medical School. Presentation of unpublished data at University of Missouri, Kansas City Medical School. Jan. 7, 2008.

Auzenne E, Ghosh S C, Khodadadian M, Rivera B, Farquhar D, Price R E, Ravoori M, Kundra V, Freedman R S, Klostergaard J (June 2007). Hyaluronic acid-paclitaxel: antitumor efficacy against CD44(+) human ovarian carcinoma xenografts. Neoplasia (New York, N.Y.) 9 (6): 479-86.

Preziosi, Luigi (2003). Cancer Modelling and Simulation. Boca Raton: CRC Press. ISBN 1-58488-361-8.

Ganguly R, Puri I K (February 2006). Mathematical model for the cancer stem cell hypothesis. Cell proliferation 39 (1): 3-14.

Wang Y, Yang J, Zheng H, Tomasek G J, Zhang P, McKeever P E, Lee E Y, Zhu Y (June 2009). Expression of mutant p53 proteins implicates a lineage relationship between neural stem cells and malignant astrocytic glioma in a murine model. Cancer Cell 15 (6): 514-26.

Clarke M F, Dick J E, Dirks P B, Eaves C J, Jamieson C H, Jones D L, Visvader J, Weissman I L, Wahl G M (October 2006). Cancer stem cells—perspectives on current status and future directions: AACR Workshop on cancer stem cells. Cancer research 66 (19): 9339-44.

Gupta P B, Onder T T, Jiang G, Tao K, Kuperwasser C, Weinberg R, Lander E S. (August 2009). Cell—Identification of Selective Inhibitors of Cancer Stem Cells by High-Throughput Screening. Cell 138(4): 645-659.

Haupt Y, Bath M L, Harris A W, Adams J M (November 1993). bmi-1 transgene induces lymphomas and collaborates with myc in tumorigenesis. Oncogene 8 (11): 3161-4.

Park I K, Qian D, Kiel M, Becker M W, Pihalja M, Weissman I L, Morrison S J, Clarke M F (May 2003). Bmi-1 is required for maintenance of adult self-renewing haematopoietic stem cells. Nature 423 (6937): 302-5.

Molofsky A V, Pardal R, Iwashita T, Park I K, Clarke M F, Morrison S J (October 2003). Bmi-1 dependence distinguishes neural stem cell self-renewal from progenitor proliferation. Nature 425 (6961): 962-7.

Hemmati H D, Nakano I, Lazareff J A, Masterman-Smith M, Geschwind D H, Bronner-Fraser M, Kornblum H I (December 2003). Cancerous stem cells can arise from pediatric brain tumors. Proceedings of the National Academy of Sciences of the United States of America 100 (25): 15178-83.

Dontu G, Jackson K W, McNicholas E, Kawamura M J, Abdallah W M, Wicha M S (2004). Role of Notch signaling in cell-fate determination of human mammary stem/progenitor cells. Breast cancer research: BCR 6 (6): R605-15.

Dievart A, Beaulieu N, Jolicoeur P (October 1999). Involvement of Notch1 in the development of mouse mammary tumors. Oncogene 18 (44): 5973-81.

Beachy P A, Karhadkar S S, Berman D M (November 2004). Tissue repair and stem cell renewal in carcinogenesis. Nature 432 (7015): 324-31.

Zhou B P, Hung M C (June 2005). Wnt, hedgehog and snail: sister pathways that control by GSK-3beta and beta-Trcp in the regulation of metastasis. Cell cycle (Georgetown, Tex.) 4 (6):

Akiyoshi T, Nakamura M, Koga K, Nakashima H, Yao T, Tsuneyoshi M, Tanaka M, Katano M (July 2006). Gli1, downregulated in colorectal cancers, inhibits proliferation of colon cancer cells involving Wnt signalling activation. Gut 55 (7): 991-9.

Bollmann F M. The many faces of telomerase: emerging extratelomeric effects. Bioessays. 2008 August; 30(8):728-32.

O. W. Brawley, Cancer Statistics, (2009) 59: 225-275 29

Nathan C F, Hibbs J B. Role of nitric oxide in macrophage antimicrobial activity. Current Opinion in Immunol 3(1): 65-70, 1991.

Eisenstein T K, Huang D, Meissler J J, al-Ramadi B. Macrophage nitric oxide mediates immunosuppression in infectious inflammation. Immunobiol 191(4-5): 493-502, 1994.

Albina J E, Henry W I. Suppression of lymphocyte proliferation through the nitric oxide synthesizing pathway. J Surg Res 50(4): 403-9, 1991.

Tomioka H, Saito H. Characterization of immunosuppressive functions of murine peritoneal macrophages induced with various agents. J Leuk Biol 51(1): 24-31, 1992.

Metzger Z, Hoffield J T, Oppenheim J J. Macrophage-mediated suppression. I. Evidence of participation of both hydrogen peroxide and prostaglandins in suppression of murine lymphocyte proliferation. J Immunol 124(2): 983-8, 1980.

Granger D: Macrophage production of nitrogen oxides in host defense against microorganisms [review]. Res Immunol 142(7): 570-2, 1991.

Hibbs J. Synthesis of nitric oxide from L-arginine: a recently discovered pathway induced by cytokines with antitumor and antimicrobial activity. Res Immunol 142(7): 565-9, 1991.

Kwon N S, Stuehr D J, Nathan C F. Inhibition of tumor cell ribonucleotide reductase by macrophage-derived nitric oxide. J Exp Med 174(4): 761-7, 1991.

De Caterina R, Libby P, Peng H B, Thannickal V J, Rajavashisth T B, Gimbrone M A, Shin W S, Liao J K. Nitric oxide decreases cytokine-induced endothelial activation: nitric oxide selectively reduces endothelial expression of adhesion molecules and proinflammatory cytokines. J Clin Invest 96(1): 60-8, 1996.

Imai Y, Kolb H, Burkhart V. Nitric oxide production from a macrophages is regulated by arachadonic acid metabolites. Biochem Biophys Res Comm 197(1): 105-9, 1993.

Gryglewski R J. Prostacyclin and nitric oxide [review]. Acta Haem Polonica 25(2:2): 75-81, 1994.

Nathan C, Xie Q W. Nitric oxide synthases: roles, tolls and controls. Cell 78(6): 915-18, 1994.

Stuehr D J, Nathan C F. Nitric oxide: a macrophage product responsible for cytostasis and respiratory inhibition in tumor target cells. J Exp Med 169(5): 1543-55, 1989.

Mirvish SS. Role of N-nitroso compound (NOC) and N-nitrosation in etiology of gastric, esophageal, nasopharyngeal and bladder cancer and the contribution of known exposure to NOC. Cancer Let 93: 17-48, 1995.

Beckman J S, Beckman T W, Chen J, Marshall P A, Freeman B A. Apparent hydroxyl radical production by peroxynitrite: implications for endothelial injury from nitric oxide and superoxide. Proc Natl Acad Sci USA 87(4): 1620-4, 1990.

Radi R, Beckman J S, Bush K M, Freeman B A. Peroxynitrite oxidation of sulfhydryls. The cytotoxic potential of superoxide and nitric oxide. J Biol Chem 266(7): 4244-50, 1991.

Bittrich H, Matzig A K, Kraker I, Appel K E. NO₂-induced DNA single strand breaks are inhibited by antioxidative vitamins in V79 cells. Chem-Biol Interact 86(3): 199-211, 1993.

Noronha-dutra A A, Epperlein M M, Woolf N. Reaction of nitric oxide with hydrogen peroxide to produce potentially cytotoxic singlet oxygen as a model for nitric oxide-mediated killing. FEBS Lett 321(1): 59-62, 1993.

Birchall M A, Winterford C M, Allen D J, and B V Harmon. Apoptosis in normal epithelium, premalignant and malignant lesions of the oropharynx and oral cavity: a Preliminary study. Oral Oncl, Eur.J. Cancer. 31:380-383, 1995.

Ke N, Albers A, Claassen G, Yu D-h, Chatterton J E, Hu X, Meyheck B, Wong-Staal F, and Q-X Li. One-week 96 well soft agar assay for cancer target validation. BioTech. 36:826-833, 2004.

Natarajan N, Shambaugh G E, Elseth K M, Haines G K, Radosevich J A. Adaptation of the Diphenylamine (DPA) assay to a 96 well plate tissue culture format and comparison with the MTT assay. BioTechniques 17:166-171, 1994.

Radosevich, J A, Ma, Y, Lee, I., Salwen, H R, Gould, V E, and S T Rosen. Monoclonal Antibody 44-3A6 as a Probe for a Novel Antigen Found Human Lung Carcinomas with glandular differentiation. Cancer Res. 45:5805-5812, 1985.

Yarmolyuk Y R, Vesper B J, Paradise W A, Elseth K M, Tarjan G, Haines G K 3rd, Radosevich J A. Part I. Development of a model system for studying nitric oxide in tumors: high nitric oxide-adapted head and neck squamous cell carcinoma cell lines. Tumour Biol. 2011 February; 32(1):77-85. Epub 2010 Oct. 23.

Tarjan G, Haines G K 3rd, Vesper B J, Xue J, Altman M B, Yarmolyuk Y R, Khurram H, Elseth K M, Roeske J C, Aydogan B, Radosevich J A. Part II. Initial molecular and cellular characterization of high nitric oxide-adapted human tongue squamous cell carcinoma cell lines. Tumour Biol. 2011 February; 32(1):87-98. Epub 2010 Oct. 21.

Paradise W A, Vesper B J, Goel A, Waltonen J D, Altman K W, Haines G K, Radosevich J A. Nitric oxide: perspectives and emerging studies of a well known cytotoxin. Int J Mol Sci. 2010 Jul. 16; 11(7):2715-45.

Vesper B J, Elseth K M, Tarjan G, Haines G K 3rd, Radosevich J A. Long-term adaptation of breast tumor cell lines to high concentrations of nitric oxide. Tumour Biol. 2010 August; 31(4):267-75. Epub 2010 May 18.

Radosevich J A, Elseth K M, Vesper B J, Tarjan G, Haines G K 3rd. Long-Term Adaptation of Lung Tumor Cell Lines with Increasing Concentrations of Nitric Oxide Donor. The Open Lung Canc J. 2009; 2: 35-44.

Vesper B J, Altman M B, Elseth K M, Roeske J C, Radosevich J A. Predicting Patient Response to Radiation: In Vitro Cell Culture Studies Using an Intensity-Modulated Radiation Therapy (IMRT) Head & Neck Phantom. 2008.

Elseth K M, Vesper B J, Schwartz J L, Bentz B G, Haines G K 3rd, Radosevich J A. The Ability of Nitric Oxide to Induce Carcinogenesis in Normal Cells. 2008.

Vesper B J, Altman, M B, Elseth K M, Roeske J C, Haines G K 3rd, Altman K W, Bentz B G, Radosevich J A. Can Nitric Oxide Be Used as a Predictor of Radiation Response. Nov. 13, 2007.

Radosevich J A, Vesper B J, Elseth K M, Haines G K 3rd, Altman K W, Bentz B G. Comparing the Ability of Normal and Tumor Cells to Adapt to High Levels of Nitric Oxide. Nov. 13, 2007.

Radosevich J A, Yarmolyuk Y R, Elseth K M, Vesper B J, Haines G K 3rd, Bentz B G. Understanding the Role Nitric Oxide Plays in Tumor Formation and Progression. September 2007.

Vesper B J, Altman, M B, Elseth K M, Roeske J C, Haines G K 3rd, Altman K W, Bentz B G, Radosevich J A. The Ability of Nitric Oxide to Induce Carcinogenesis in Normal Cells. Nov. 13, 2007.

Vesper B J, Hammer N D, Bentz B G, Haines G K 3rd, Radosevich J A. Predicting Radiosensitivity: Correlation of Nitric Oxide Synthase and GST-π Expression in Human Oral Cancers. Sep. 24-28, 2005.

What is claimed is:

1. A method of obtaining tumor stem cells comprising
culturing a cell population, and
exposing the cultured cell population to free radicals to obtain the tumor stem cells.

2. The method of claim 1 wherein the step of exposing the cultured cell population to free radicals comprises exposing the cultured cell population to nitrogen-based free radicals.

3. The method of claim 2 wherein nitrogen-based free radicals includes nitric oxide (NO).

4. The method of claim 2 wherein nitrogen-based free radicals includes a NO donor.

5. The method of claim 4 wherein the step of exposing the cultured cell population further comprises exposing the population to Diethylenetriamine NONOate (DETA NONOate).

6. The method of claim 1 wherein the step of exposing the cultured cell population further comprises increasing NO synthase in the population.

7. The method of claim 1 wherein the step of exposing the cultured cell population to free radicals comprises exposing the cultured cell population to oxygen-based free radicals.

8. The method of claim 7 wherein oxygen-based free radicals includes hydrogen peroxide.

9. The method of claim 1 wherein the step of exposing the cultured cell population to free radicals comprises exposing the cultured cell population to increasing levels of free radicals

10. The method of claim 1 wherein the cell population is a tumor cell population.

11. The method of claim 10 wherein the method further comprises isolating tumor stem cells by exposing the tumor cell population to a level of free radicals sufficient to selectively kill tumor cells but not tumor stem cells.

12. The method of claim 10 wherein the method further comprises inducing tumor stem cells present in the tumor cell population to expand.

13. The method of claim 10 wherein the method further comprises inducing dedifferentiation of the tumor cells into tumor stem cells.

14. The method of claim 10 wherein the method further comprises selecting tumor stem cells from the tumor cell population.

15. The method of claim 1 further comprising isolating tumor stem cells.

16. The method of claim 15, wherein the step of isolating the tumor stem cells includes exclusion of tumor stem cells by a vital dye staining.

17. The method of claim 16 wherein the vital dye is Hoechst 33342.

18. The method of claim 15, wherein the step of isolating the tumor stem cells includes measuring expression of aldehyde dehydrogenase (ALDH).

19. The method of claim 15, wherein the step of isolating the tumor stem cells includes assaying DNA tails in a COMET assay.

20. The method of claim 15, wherein the step of isolating the tumor stem cells includes measuring regulation of a DNA repair enzyme.

21. The method of claim 20 wherein the DNA repair enzyme is an apurinic/apyrimidinic endonuclease-1 (APE-1) DNA repair enzyme.

22. The method of claim 1 wherein the step of culturing the cell population comprises culturing cells comprising at least one population of cells selected from the group consisting of normal cells, non-tumor cells, cell lines, primary tissues, and immortalized cells.

23. A high nitric oxide (HNO) tumor stem cell exhibiting at least one of the following characteristics:
cell surface expression of at least the cell surface markers CD38 and CD166;
increased expression of aldehyde dehydrogenase (ALDH); and
upregulation of at least one DNA repair enzyme.

24. The HNO tumor cells of claim 23, wherein the HNO tumor stem cell is further characterized by at least one of the following:
resistance to DNA fragmentation;
growth in high free radical environments;
resistance to UV and gamma radiation; and
temperature insensitivity.

25. The HNO tumor stem cell of claim 23 further comprises a gene expression profile as shown in Appendix A.

26. The HNO tumor stem cell of claim 23 further comprises a gene expression profile at least 50% homologous to the expression profile as shown in Appendix A.

27. The HNO tumor stem cell of claim 23 further comprises a gene expression profile at least 50% homologous to a subset of genes in the expression profile as shown in Appendix A.

28. The HNO tumor stem cell of claim 27, wherein the subset of genes includes at least 5 or more genes.

29. A high nitric oxide (HNO) tumor stem cell obtained by the method of claim 1.

30-49. (canceled)
